(19) United States
(12) Patent Application Publication
(10) Pub. No.: US 2026/0283507 A1
(43) Pub. Date: Sep. 24, 2026

(54) Methods and Systems for Identifying Gait Biomarkers Used to Drive Adaptive Deep Brain Stimulation

(71) Applicant: The Regents of the University of California, Oakland, CA (US)

(72) Inventors: Doris Wang, San Francisco, CA (US); Kenneth Louie, San Francisco, CA (US)

(21) Appl. No.: 19/475,978

(22) PCT Filed: Apr. 29, 2024
(86) PCT No.: PCT/US2024/026853

§ 371 Date: Oct. 16, 2025

Related U.S. Application Data
(60) Provisional application No. 63/462,930, filed on Apr. 28, 2023.
(60) Provisional application No. 63/465,385, filed on May 10, 2023.

(51) Int. Cl. A61B 5/11; A61B 5/00; A61B 5/37

(52) U.S. Cl. CPC A61B 5/112; A61B 5/37; A61B 5/4082; A61B 5/7267

(57) ABSTRACT

The inventors discovered that neural oscillatory activities in the sensorimotor cortex region of the brain and the basal ganglia system of the brain are indicative of physiological gait events. The invention utilizes this discovery, along with recent advances in neural interfaces and machine learning techniques, to provide new and useful methods for identifying gait events directly from the neural activity of an individual. In particular, methods and systems are provided that produce trained classification models capable of accurately identifying left and right leg events during walking. Trained classification models exhibiting above a threshold level of accuracy may then be used to generate control signals for real time adaptive deep brain stimulation in order to improve gait function in patients with Parkinson's disease.

### CROSS-REFERENCE

Pursuant to 35 U.S.C. § 119 (e), this application claims priority to the filing date of U.S. Provisional Patent Application Ser. No. 63/462,930 filed on Apr. 28, 2023, and the filing date of U.S. Provisional Patent Application Ser. No. 63/465,385 filed on May 10, 2023, the disclosure of which applications are herein incorporated by reference.

### INTRODUCTION

Human walking is a complex motor task that requires the flexible coordination of reciprocal left and right leg movements. Natural upright walking consists of each leg alternating between the stance phase, when the foot is in contact with the ground, and the swing phase, when the foot is in the air; these two phases make up the “gait cycle,” comprised of a series of stereotyped events such as left and right heel-strikes and toe-offs.

The basal ganglia and motor cortical areas may be nodes of the supraspinal network that regulate human gait, given their respective connectivity to each other, locomotor regions in the midbrain and the brain stem. Understanding of the cortico-basal ganglia network activities that underlie natural walking in humans is, however, limited due to methodological constraints. Scalp electroencephalography (EEG) studies have shown that natural overground walking is associated with fluctuations in the alpha (8-12 Hz), beta (13-30 Hz), and gamma (70-90 Hz) frequency ranges from the sensorimotor regions of healthy subjects. However, EEG lacks the spatial resolution to discern whether these rhythms originate from the motor cortex or represent sensory feedback during walking and are prone to movement artifacts. In addition, very little is known about the role of the basal ganglia in the regulation of gait. In some instances, electrical stimulation or lesioning of the subthalamic nucleus (STN) or globus pallidus interna (GPi) of the basal ganglia can induce gait abnormalities such as freezing of gait. Furthermore, little is known about cortical-basal ganglia interactions during the natural gait cycle.

### SUMMARY

The inventors discovered that neural oscillatory activities in the sensorimotor cortex region of the brain and the basal ganglia system of the brain are indicative of physiological gait events. The invention utilizes this discovery, along with recent advances in neural interfaces and machine learning techniques, to provide new and useful methods for identifying gait events directly from the neural activity of an individual. In particular, methods and systems are provided that produce trained classification models capable of accurately identifying left and right leg events during walking. Trained classification models exhibiting above a threshold level of accuracy may then be used to generate control signals for real time adaptive deep brain stimulation in order to improve gait function in patients with Parkinson's disease.

In one aspect, methods of identifying neural activity biomarkers of alternating bilateral movement in a subject are provided. Aspects of the methods include: positioning a neural recording device including one or more electrodes at a location in a sensorimotor cortex region or basal ganglia system of the brain of the subject to record brain electrical signal data associated with the subject performing the alternating bilateral movement; instructing the subject to perform an activity including the alternating bilateral movement; recording the brain electrical signal data associated with the movement performed by the subject using the neural recording device, wherein the brain electrical signal data is transmitted to a processor; recording one or more body parts of the subject performing the movement using a sensor, wherein the movement data recorded by the sensor is transmitted to the processor; processing the brain electrical signal data and the movement data using the processor, wherein the processing includes synchronizing the brain electrical signal data and the movement data; identifying one or more neural activity biomarkers of the movement in the subject from the processed brain electrical signal data and movement data using the processor, wherein the processor is programmed to use a machine learning algorithm for the identification.

In certain embodiments, the alternating bilateral movement includes the repeated abduction, adduction, flexion, extension, and/or circumduction of one or more body parts of the subject. In some embodiments, the movement includes the repeated flexion and extension of one or more body parts of the subject such as one or more of the subject's arms or legs. In some embodiments, the movement includes walking.

In certain embodiments, the neural recording device includes two or more electrodes. In some embodiments, one or more of the electrodes are positioned at a location in a sensorimotor cortex region of the brain and the basal ganglia system of the brain. In some embodiments, one or more of the electrodes are positioned at a location in both the left and right sensorimotor cortex region of the brain and/or both the left and right basal ganglia system of the brain. In some embodiments, the sensorimotor cortex region of the brain includes the precentral gyrus, or premotor cortex, or supplemental motor area. In some embodiments, the sensorimotor cortex region of the brain includes the somatosensory cortex. In some embodiments, the basal ganglia system includes the subthalamic nucleus or the globus pallidus.

In certain embodiments, the neural recording device includes a deep brain stimulation lead configured to be positioned in the basal ganglia system of the brain. In some embodiments, the neural recording device includes bilateral leads. In some embodiments, the neural recording device includes an electrocorticography (ECoG) paddle configured to be positioned in the sensorimotor cortex region of the brain. In some embodiments, the electrocorticography paddle includes bilateral paddles. In some embodiments, the neural recording device includes bilateral paddles placed elsewhere on the cortex of the brain.

In certain embodiments, the processing further includes calculating one or more of a continuous wavelet transform (CWT), wavelet coherence, short-time Fourier transform (STFT), and power spectral density (PSD) using the synchronized brain electrical signal data and the movement data.

In certain embodiments, the processor is programmed to use a machine learning algorithm including a Random Forest (RF) or Recursive Feature Elimination (RFE) feature selection algorithm and a machine learning model. In some embodiments, the processed brain electrical signal data and movement data are used to train the feature selection algorithm for identifying brain electrical signal data features of neural activity biomarkers of alternating bilateral movement in the subject. In some embodiments, the feature selection algorithm (e.g., the trained feature selection algorithm) is used to train a machine learning model used for alternating bilateral movement event classification from recorded brain electrical signal data. In some embodiments, the machine learning algorithm includes one or more of a K-nearest neighbors (KNN), logistic regression, linear discriminant analysis (LDA), Gradient Boosted Decision Trees (XGBoost), and neural network algorithm for training one or more machine learning models of an ensemble of models for event classification.

In certain embodiments, the trained ensemble of machine learning models is used to generate control signals for real time adaptive deep brain stimulation. In some embodiments, the identified neural activity biomarkers of alternating bilateral movement are used to generate control signals for real time adaptive deep brain stimulation.

In another aspect, a system for identifying neural activity biomarkers of alternating bilateral movement in a subject from neural activity is provided, the system including: a neural recording device including an electrode adapted for positioning at a location in a sensorimotor cortex region or basal ganglia system of the brain of the subject to record brain electrical signal data associated with the subject performing the alternating bilateral movement; a sensor configured to record one or more body parts of the subject performing the movement; a processor configured to receive the brain electrical signal data from the neural recording device and the movement data from the sensor; and memory operably coupled to the processor wherein the memory includes instructions stored thereon, which when executed by the processor, cause the processor to process the brain electrical signal data and the movement data by synchronizing the brain electrical signal data and the movement data, and identify one or more neural activity biomarkers of the movement in the subject from the processed brain electrical signal data and movement data using a machine learning algorithm.

In another aspect, a kit including a system described herein and instructions for using the system for recording and decoding brain electrical signal data associated with an alternating bilateral movement performed by a subject is provided.

### BRIEF DESCRIPTION OF THE FIGURES

FIGS. 1A to 1B: DBS and cortical lead localization. (A) 3D reconstructions of all DBS lead locations in the STN. (B) 3D reconstructions of cortical electrode paddle location.

FIGS. 2A to 2C: Synchronized gait kinematic data with raw local field potential recordings during natural walking. (A) Illustration of gait events and phases during a single gait cycle, aligned to left heel-strike. (B) Heel-strike and toe-off gait events detected from the left and right force sensitive resistor data. (C) Example local field potential recordings from both STN and M1 synchronized to a gait cycle.

FIGS. 3A to 3B: Grand average z-score spectrograms from the ventral (A) and dorsal (B) STNs normalized to a gait cycle.

FIGS. 4A to 4B: Grand average z-score coherogram from STN-M1 and -S1 normalized to a gait cycle. (A) STN-M1 coherogram. (B) STN-S1 coherogram.

FIGS. 5A to 5C: Unique frequency bands within each subject can differentiate gait events. (A) Average heel-strike and toe-off PSDs from the STN and M1. (B) Average power and standard error±1 second around the gait event. (C) Boxplot of gait event power within the frequency bands from FIG. 5B.

FIG. 6: Results of gait event decoding using oscillatory features, each subject's models are shown on each row.

FIGS. 7A to 7B: Depicts cortical local field potentials demonstrating spectral power modulations during the gait cycle. (A) Left M1 and right M1 field potentials. (B) Left S1 and right S1 field potentials.

FIGS. 8A to 8B: Spectrograms of a single gait cycle from the STN and sensorimotor cortices from all recorded areas from all subjects in the study.

FIG. 9: Depicts a visualization of the arbitrary length frequency bands created and an ANOVA p-value heat-map.

FIG. 10: Shows results from classifier models built using coherence values between the STN and M1.

FIG. 11: Provides a flow diagram depicting a method of slow adaptive DBS for gait accordance with an embodiment of the invention.

FIG. 12: Provides a flow diagram depicting a method of fast adaptive DBS for gait in accordance with an embodiment of the invention.

FIG. 13: Depicts subcortical and cortical lead reconstructions for five patients having DBS leads targeting the globus pallidus internus (GPi).

FIGS. 14A to 14B: Provide an example of gait phase-specific modulation in the GPi and cortical regions of a subject.

FIGS. 15A to 15B: Provide an example of adaptive deep brain stimulation therapy in a subject.

### DETAILED DESCRIPTION

Methods for identifying alternating bilateral movement events directly from the neural activity of an individual are provided. Aspects of the methods include: positioning a neural recording device including one or more electrodes at a location in a sensorimotor cortex region or basal ganglia system of the brain of the subject to record brain electrical signal data associated with the subject performing the alternating bilateral movement; instructing the subject to perform an activity including the alternating bilateral movement; recording the brain electrical signal data associated with the movement performed by the subject using the neural recording device, wherein the brain electrical signal data is transmitted to a processor; recording one or more body parts of the subject performing the movement using a sensor, wherein the movement data recorded by the sensor is transmitted to the processor; processing the brain electrical signal data and the movement data using the processor, wherein the processing includes synchronizing the brain electrical signal data and the movement data; identifying one or more neural activity biomarkers of the movement in the subject from the processed brain electrical signal data and movement data using the processor, wherein the processor is programmed to use a machine learning algorithm for the identification. Also provided are systems for use in practicing methods of the invention.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, methods for identifying alternating bilateral movement events directly from the neural activity of an individual are provided. Aspects of the methods include: positioning a neural recording device including one or more electrodes at a location in a sensorimotor cortex region or basal ganglia system of the brain of the subject to record brain electrical signal data associated with the subject performing the alternating bilateral movement; instructing the subject to perform an activity including the alternating bilateral movement; recording the brain electrical signal data associated with the movement performed by the subject using the neural recording device, wherein the brain electrical signal data is transmitted to a processor; recording one or more body parts of the subject performing the movement using a sensor, wherein the movement data recorded by the sensor is transmitted to the processor; processing the brain electrical signal data and the movement data using the processor, wherein the processing includes synchronizing the brain electrical signal data and the movement data; identifying one or more neural activity biomarkers of the movement in the subject from the processed brain electrical signal data and movement data using the processor, wherein the processor is programmed to use a machine learning algorithm for the identification.

### The Alternating Bilateral Movement

As described above, embodiments of the methods include instructing the subject to perform an activity including an alternating bilateral movement and recording one or more body parts performing the movement as well as brain electrical signal data associated with the subject performing the movement. The activity performed by the subject may be any activity including an alternating movement of the left- and right-hand side of the body. The sensor may be configured to record any physical property associated with the real-time movement of the one or more body parts performing the alternating bilateral movement.

The terms “subject”, “individual”, “patient”, and “participant” are used interchangeably herein and refer to a patient having a neurological disorder. The patient is preferably human, e.g., a child, an adolescent, an adult, such as a young, middle-aged, or elderly human who may benefit from the systems and methods disclosed herein for improving movement function. In some embodiments, the subject performing the alternating bilateral movement may have a neurological disorder affecting movement. In some cases, the neurological disorder may include any neurological disorder with the potential to be treated with deep brain stimulation (DBS). For example, the neurological disorder may include essential tremor, Parkinson's disease (PD), dystonia and/or Huntington's disease.

In some cases, the neurological disorder may affect or increase the difficulty the subject experiences in performing the movement. In some embodiments, the neurological disorder may be a degenerative disorder. In these instances, the subject may be at any stage of progression of the disorder. For example, the neurological disorder may include Parkinson's disease and the subject may have early PD (i.e., stage 1 or 2 PD), middle PD (i.e., stage 3 PD), or advanced PD (i.e., stage 4 or 5 PD). In some embodiments, the subject may perform the alternating bilateral movement in order to train one or more machine learning models to identify and/or classify right and left movement events (e.g., as described in greater detail below) when the subject is at an earlier PD stage (i.e., stage 1 or 2 PD). The trained machine learning model(s) may then be utilized to generate control signals for real time adaptive DBS (aDBS) from the neural activity of the subject when the subject has progressed to a later stage of PD (i.e., stage 3-5 PD) in order to improve movement function in the subject. In embodiments where the subject has PD, the subject may be on a medication for the treatment of PD such as, e.g., a dopaminergic medication.

As described above, the activity performed by the subject may be any activity including an alternating movement of the left- and right-hand side of the body. In other words, the activity may be an alternating bilateral movement that is repeated by equivalent body parts on both the left and right sides of the body. Activities including an alternating bilateral movement, in accordance with embodiments of the methods, may vary and include, but are not limited to, those found below. In some embodiments, the alternating bilateral movement may include the movement of one or more body parts of the subject. The one or more body parts may include, but are not limited to, the subject's arms, legs, hands, pelvis, hips, back, neck, ankles, feet, hands, phalanges, or shoulders. In some embodiments, the one or more body parts includes a joint of the subject such as, e.g., a ball and socket joint, saddle joint, hinge joint, condyloid joint, pivot joint, or gliding joint.

In some embodiments, the alternating bilateral movement may include a repeated abduction, adduction, flexion, extension, or circumduction of the one or more body parts of the subject. For example, the alternating bilateral movement may include the repeated abduction and adduction of one or both of the subject's leg joints. In some embodiments, the alternating bilateral movement includes walking, jogging, or running. In some instances, the alternating bilateral movement includes walking.

In some embodiments, the alternating bilateral movement is repeated two or more times, such as five or more times, or ten or more times, or twenty or more times, or fifty or more times, or one hundred or more times. In some embodiments, the alternating bilateral movement is repeated for a set amount of time. For example, the alternating bilateral movement may be repeated for five seconds or more, such as ten seconds or more, or twenty seconds or more, or thirty seconds or more, or sixty seconds or more, or one hundred seconds or more. In some embodiments, the alternating bilateral movement is repeated a number of times sufficient to accurately identify neural activity biomarkers of the alternating bilateral movement in the subject as discussed in greater detail below.

The manner in which the subject is instructed or notified to begin or cease performing the activity may vary and may include any number of visual or auditory means. In some embodiments, a screen (such as, e.g., a TV screen or a computer monitor) may notify the subject to begin performing the activity. In some cases, an auditory signal, such as a beep or a voice, may notify the subject to begin performing the activity.

Embodiments of the methods include recording one or more body parts of the subject performing the movement using a sensor, wherein the movement data recorded by the sensor is transmitted to the processor. As described above, the sensor may be configured to record any physical property associated with the real-time movement of the one or more body parts performing the alternating bilateral movement. In some cases, the sensor may be a kinematics sensor. In some embodiments, the sensor is a wearable sensor or includes a wearable component. In some embodiments, the sensor is configured to record one or more of: the acceleration or velocity of the one or more body parts; the force generated by the one or more body parts; and the angle between the one or more body parts and a reference body part.

In some embodiments, the sensor is configured to record the acceleration or velocity of the one or more body parts. In these instances, the sensor may include one or more accelerometers (e.g., wearable accelerometers), inertial measurement unit sensors, video or image sensors, or laser emitters. In embodiments where the sensor includes a video or image sensor (e.g., a camera or a camcorder), the subject may wear a motion tracking marker. In embodiments where the sensor includes a laser emitter, the emitted laser may be, e.g., an infrared (IR) laser beam, a near infrared (NIR) laser beam, or a laser beam of visible light. In these instances, the laser emitter may be part of a Light Detection and Ranging (LiDAR) scanner used to determine the distance one or more body parts of the subject is from a reference point. In some cases, the reference point may be another body part of the subject.

In some embodiments, the sensor is configured to record the force generated by the one or more body parts. In these instances, the sensor may be a force sensitive resistor. In embodiments where the alternating bilateral movement includes walking and the sensor includes a force sensitive resistor, the sensor may be configured to measure the heel force and/or toe force generated by the subject as they walk.

In some embodiments, the sensor is configured to record the angle between the one or more body parts and a reference body part. In these instances, the sensor may be a goniometer. In embodiments where the alternating bilateral movement includes walking and the sensor includes a goniometer, the sensor may be placed next to the lateral malleolus.

In some embodiments, the sensor includes an electromyography (EMG) sensor. In these instances, the EMG sensor may be configured to measure electrical activity from one or more of the muscles driving the alternating bilateral movement of the subject. In some cases, the EMG sensor may be configured to measure electrical activity from any muscle that moves during the alternating bilateral movement performed by the subject. In some embodiments, the EMG sensor is a surface EMG sensor and/or is wearable.

As described above, the movement data recorded by the sensor is transmitted to the processor. In some embodiments, the movement data is electronic movement data (such as, e.g., digital movement data) and the electronic movement data is transmitted electronically. In these embodiments, the movement data may be transmitted from the sensor to the processor through a variety of means. In some embodiments, the sensor may transmit the movement data directly, e.g., through a wire. In other embodiments, the sensor may transmit the movement data by converting electronic movement data into an electromagnetic or ultrasound wave. For example, the sensor may transmit the movement data to the processor using Bluetooth®, Wi-Fi, Global System for Mobile communications (GSM), etc.

As described above, embodiments of the methods may include instructing the subject to perform an activity including an alternating bilateral movement and recording one or more body parts performing the movement as well as brain electrical signal data associated with the subject performing the movement. The movement data, along with the brain electrical signal data, is then transmitted to a processor where it is processed for further use to generate neural activity biomarkers of alternating bilateral movement in the subject, as described in greater detail below.

### Recording Brain Electrical Signal Data

As described above, embodiments of the methods include positioning a neural recording device including one or more electrodes at a location in a sensorimotor cortex region and/or basal ganglia system of the brain of the subject to record brain electrical signal data (i.e., local field potentials) associated with the subject performing the alternating bilateral movement. In some embodiments, the neural recording device includes two or more electrodes configured to be positioned at two or more different locations of the brain.

Positioning an electrode for recording brain activity at specified region(s) of the brain may be carried out using standard surgical procedures for placement of intra-cranial electrodes. As used herein, the phrases “an electrode” or “the electrode” refer to a single electrode or multiple electrodes such as an electrode array. As used herein, the term “contact” as used in the context of an electrode in contact with a region of the brain refers to a physical association between the electrode and the region. In other words, an electrode that is in contact with a region of the brain is physically touching the region of the brain. An electrode in contact with a region of the brain can be used to detect electrical signals corresponding to neural activity associated with the alternating bilateral movement (e.g., walking) performed by the subject. Electrodes used in the methods disclosed herein may be monopolar (cathode or anode) or bipolar (e.g., having an anode and a cathode).

In certain embodiments, one or more electrodes are used to record electrical signals for neural activity associated with an alternating bilateral movement (e.g., walking) performed by the subject in one or more brain regions. An electrode may be placed, for example, in a region of the sensorimotor cortex involved in movement such as the primary motor cortex (M1) (including the precentral gyrus such as, e.g., the hand knob area of the precentral gyrus), the somatosensory cortex (S1) and/or the premotor area. In some embodiments, an electrode may be placed in a region of the primary motor cortex (M1) and/or the somatosensory cortex (S1). An electrode may alternatively or additionally be placed in the basal ganglia system of the brain including, e.g., the subthalamic nucleus (STN). In certain cases, placing the electrode may involve positioning the electrode on the surface of the specified region(s) of the brain. In some embodiments, placing the electrode may involve positioning the electrode in the subdural space over the specified region(s) of the brain. In some cases, placing the electrode may involve positioning the electrode into the specified region(s) of the brain (e.g., when the specified region is the STN).

The precise number of electrodes contained in the neural recording device may vary. In certain aspects, the neural recording may include 2 or more electrodes, such as 3 or more, 4 or more, 5 or more, 10 or more, 20 or more, 50 or more, 100 or more, including 200 or more, e.g., about 2 to 4 electrodes, about 4 to 10 electrodes, about 10 to 20 electrodes, about 20 to 50 electrodes, about 50 to 100 electrodes, or more electrodes. The electrodes may be arranged into a regular repeating pattern (e.g., a grid), or no pattern. An electrode that conforms to the target site for optimal recording of electrical signals from neural activity associated with an alternating bilateral movement performed by a subject may be used.

In certain embodiments, the method further includes mapping the brain of the subject to optimize positioning of an electrode. Positioning of an electrode may be optimized to detect brain activity features associated with an alternating bilateral movement (e.g., walking) performed by the subject and to achieve optimal decoding of the performed movement. For example, patterns of electrical signals in specific frequency ranges (e.g., alpha, delta, beta, gamma, and/or high gamma) may be used for detecting an alternating bilateral movement performed by the subject and decoding neural activity biomarkers of the alternating bilateral movement in the subject. Thus, electrodes may be positioned to optimize detection and/or decoding of brain activity in specific frequency ranges to improve movement (e.g., gait) function in patients with a neurological disorder such as patients having PD.

In certain aspects, the methods and systems of the present disclosure may include recording brain activity, for example, electrical activity in the ventral and/or dorsal STN or the M1 or S1 regions, where patterns of alpha through gamma-frequency neural activity or lower frequencies associated with an alternating bilateral movement performed by the subject such as, e.g., a specific muscle or muscle group of the subject may be detected. In certain cases, electrical activity from a plurality of locations in the brain may be measured. In some embodiments, one or more locations in a sensorimotor cortex region of the brain and/or the basal ganglia system of the brain may be measured. In some cases, one or more locations in both the left and right sensorimotor cortex region of the brain and/or both the left and right basal ganglia system of the brain may be measured. In some embodiments, the sensorimotor cortex region of the brain includes the M1 region such as, e.g., the hand knob area of the M1 region. In some embodiments, the sensorimotor cortex region of the brain includes the S1. In some embodiments, the basal ganglia system includes the STN such as, e.g., the dorsal and/or ventral STN.

Electrical activity in a wide frequency band (0 Hz to 200 Hz) may be measured in one or more regions of the STN and/or sensorimotor cortex. In some embodiments, electrical activity in a range from about 0 Hz to 200 Hz, such as about 0 Hz to 100 Hz, or about 0 Hz to 50 Hz, or about 4 Hz to 8 Hz, or about 8 Hz to 10 Hz, or about 4 Hz to 12 Hz, or about 8 Hz to 12 Hz, or about 5 Hz to 15 Hz, or about 5 Hz to 23 Hz, or about 4 Hz to 30 Hz, or about 8 Hz to 30 Hz, or about 13 Hz to 30 Hz, or about 35 Hz to 45 Hz, or about 2.5 Hz to 50 Hz, or about 10 Hz to 50 Hz, or about 50 Hz to 200 Hz may be measured in a region of the STN and/or sensorimotor cortex.

In some embodiments, electrical activity in a wide frequency band (0 Hz to 50 Hz) in the ventral STN and/or in low frequency band (5 Hz to 15 Hz) in the dorsal STN may be measured. In some embodiments, electrical activity in the beta frequency range (such as 13 Hz to 30 Hz) may be measured in a region of the STN. In some embodiments, electrical activity in the high gamma frequency range (such as 50 Hz to 200 Hz) may be measured in a region of the STN. In some embodiments, electrical activity in a wide frequency band (0 Hz to 50 Hz), in the theta-alpha frequency range (such as 5 Hz to 12 Hz) and/or in the high frequency gamma range (50 Hz to 200 Hz) is measured in the left and/or right M1, the left and/or right S1, and/or the left and/or right STN (e.g., ventral and/or dorsal).

Detection of brain activity may be performed by any method known in the art. For example, functional brain imaging of neural activity may be carried out by electrical methods such as electrocorticography (ECOG), electroencephalography (EEG), stereoelectroencephalography (sEEG), magnetoencephalography (MEG), single photon emission computed tomography (SPECT), as well as metabolic and blood flow studies such as functional magnetic resonance imaging (fMRI), positron emission tomography (PET), functional near-infrared spectroscopy (fNIRS), and time-domain functional near-infrared spectroscopy. In some embodiments, the neural recording device includes a lead configured to be positioned in the basal ganglia system of the brain (e.g., the left and/or right STN). In some embodiments, the neural recording device includes bilateral leads such as, e.g., bilateral DBS leads. In some embodiments, the neural recording device includes an ECoG paddle configured to be positioned in the sensorimotor cortex region of the brain (e.g., the left and/or right M1, the left and/or right S1, etc.). In these instances, the neural recording device may include bilateral paddles.

Embodiments of the methods may include implanting a pulse generator in or on the body of the subject for transmitting the brain electrical signal data to the processor such as, e.g., over the pectoralis muscles of the subject. In some embodiments, the pulse generator utilizes a wireless communication protocol using an electromagnetic carrier wave (e.g., a radio wave, microwave, or an infrared carrier wave) or ultrasound to transfer data from the neural recording device to the processor. Neural recording devices (e.g., neural recording paddles) may be commercially available devices (e.g., Medtronic model 0913025) and may include commercially available DBS leads (e.g., Medtronic model 33015 or 33005). Pulse generators may be commercially available generators such as, e.g., Medtronic B35200 Percept PC. The processor may be provided by a computer or a handheld computing device (e.g., cell phone or tablet) programmed to identify one or more neural activity biomarkers of the movement in the subject from the recorded brain electrical signal data.

As described above, embodiments of the methods may include positioning a neural recording device including one or more electrodes at a location in a sensorimotor cortex region and/or basal ganglia system of the brain of the subject to record brain electrical signal data associated with the subject performing the alternating bilateral movement. The brain electrical signal data, along with the movement data as described above, is then transmitted to a processor where it is processed for further use, as described in greater detail below.

### Data Processing and Machine Learning Techniques

As described above, embodiments of the methods include processing the brain electrical signal data and the movement data using the processor, wherein the processing includes synchronizing the brain electrical signal data and the movement data. Embodiments of the methods also include identifying one or more neural activity biomarkers of the movement in the subject from the processed brain electrical signal data and movement data using the processor, wherein the processor is programmed to use a machine learning algorithm for the identification.

In some embodiments, processing the brain electrical signal data and the movement data generated, e.g., as described above includes synchronizing the brain electrical signal data and the movement data. In some cases, the pulse generator utilized to transmit the brain electrical signal data to the processor further includes an accelerometer. In these cases, the brain electrical signal data and the movement data may be synchronized by aligning the acceleration peaks of the pulse generator and the acceleration peaks of the movement sensor(s). In some cases, the processing further includes preprocessing the brain electrical signal data using high-pass and/or low-pass filters.

In some embodiments, one or more signal processing techniques may be used to process the synchronized brain electrical signal data and the movement data. In these embodiments, one or more signal processing techniques may be used to analyze the coherence between brain electrical signal data obtained from two or more different regions of the brain. In some cases, the one or more signal processing techniques may be used to analyze frequency bands where power (i.e., amplitude) differed among alternating bilateral movement events (such as, e.g., specific portions of the movement performed by the left- or right-hand side of the body). In some embodiments, the processing techniques may include calculating one or more of a continuous wavelet transform (CWT), wavelet coherence, short-time Fourier transform (STFT), and power spectral density (PSD) using the synchronized brain electrical signal data and the movement data.

In some embodiments, the processed brain electrical signal data and movement data is used by a processor to identify neural activity biomarkers of alternating bilateral movement in a subject using machine learning techniques. The terms “neural activity biomarker”, “alternating bilateral movement biomarker”, and any relevant variation of “alternating bilateral movement biomarker” (e.g., “gait biomarker” when the “alternating bilateral movement biomarker” is walking) are used interchangeably herein. By neural activity biomarkers (i.e., of alternating bilateral movement) is meant one or more features of the brain electrical signal data that may be used as a measurable indicator of the present phase of the alternating bilateral movement. For example, power (i.e., amplitude) modulations of neural activity in a specific frequency range of a specific region of the brain may indicate the stance or swing phase of the left or right leg during walking. In some embodiments, the phase of the alternating bilateral movement may be characterized by an alternating bilateral movement event. For example, when the alternating bilateral movement is walking, the event may include a left or right heel strike. In some embodiments, the biomarker may simply be a measurable indicator that the alternating bilateral movement is being performed. For example, when the alternating bilateral movement includes walking, one or more neural activity biomarkers may be used to determine if the subject is currently walking or is standing still or sitting. In some cases, the identified neural activity biomarkers of alternating bilateral movement in the subject are used to classify segments of brain electrical signal data as indicative of an alternating bilateral movement event (e.g., using a trained classification machine learning model as discussed below).

Machine learning techniques employed for identifying one or more neural activity biomarkers of the alternating bilateral movement in the subject from the processed brain electrical signal data and movement data and/or for classifying alternating bilateral movement events (e.g., using identified neural activity biomarker(s)) may vary and may include, but are not limited to, any of the techniques discussed below or any standard machine learning technique, as well as combinations thereof, as is known in the art. In some embodiments, the machine learning techniques may include training a machine learning model using the processed brain electrical signal data and movement data and a machine learning algorithm. The machine learning model, in accordance with embodiments of the methods, may vary and may include, but is not limited to, any of the algorithms as discussed below. In some embodiments, the training may further include validating and testing the machine learning model.

In some embodiments, the machine learning model may include, or be configured to employ, a feature selection algorithm for identifying brain electrical signal data features of neural activity biomarkers. In some instances, the feature selection algorithm employs wrapper methods (e.g., forward, backward, and stepwise selection), filter methods (e.g., ANOVA, Pearson correlation, variance thresholding), and/or embedded methods (e.g., Lasso, Ridge, Decision Tree). In some embodiments, the feature selection algorithm used for identifying brain electrical signal data features of neural activity biomarkers is a Random Forest (RF) or Recursive Feature Elimination (RFE) algorithm. In some embodiments, the trained feature selection algorithm (e.g., a trained RF or RFE machine learning model) is one of multiple ensemble models used for alternating bilateral movement event classification (i.e., ensemble modeling/learning techniques are employed). In some embodiments, one or more of the ensemble models includes, or is configured to employ, a K-nearest neighbors (KNN), logistic regression, linear discriminant analysis (LDA), Gradient Boosted Decision Trees (XGBoost), and/or neural network (NN) algorithm for event classification. In embodiments where a NN is employed for classification, the NN may be a deep learning NN that is three or more layers deep, such as five or more layers deep, or ten or more, or twelve or more, or thirty or more, or fifty or more, or one hundred or more. In some embodiments, the NN may include a convolutional neural network (CNN), recurrent neural network (RNN), or may include transformer architecture. In some embodiments, the ensemble of models used for classification includes an RF or RFE model and an LDA model. In some instances, the ensemble of models used for classification consists of an RF or RFE model and an LDA model.

Training may depend on the nature or architecture of the machine learning model. For example, in embodiments where the machine learning model includes ensemble models, training may include bagging or bootstrap aggregating. In some embodiments, training may be supervised using the brain electrical signal data aligned or synchronized with the movement data as discussed above. In some embodiments, the model training algorithms and hyperparameters used to control the training may depend on, e.g., the nature or architecture of the machine learning model, the tasks the machine learning model is trained to perform, the desired accuracy or efficiency of the machine learning model, and/or the nature or size of the training data set. In some cases, algorithms or techniques are used during training that correct for overfitting issues

In some embodiments, the training may further include testing the trained machine learning model or machine learning models. By testing in this context is meant evaluating the trained machine learning model using brain electrical signal data synchronized with movement data different from the brain electrical signal data synchronized with movement data used for training after the machine learning model has finished training. In some embodiments, a first subset of the brain electrical signal data synchronized with movement data is used for training and a second subset of the brain electrical signal data synchronized with movement data is used for testing. The testing may use one or more metrics to evaluate the performance of the trained machine learning model or machine learning models. In some cases, the metric may include a number, or percent, reflecting the accuracy of alternating bilateral movement event classification of a trained model. In some embodiments, class accuracy is calculated. In some embodiments, the metric may be used to determine if the trained machine learning model performs sufficiently using, e.g., a predetermined threshold (i.e., requirement). In these instances, if the trained machine learning model does not meet the predetermined threshold, the model may be discarded and/or another model may be trained. In embodiments where another machine learning model is trained, one or more of the model architecture, training and/or the training data set may be modified prior to training. In some instances, machine learning models are trained until a trained machine learning models meets the predetermined threshold. The division between the first and second subsets of the brain electrical signal data synchronized with movement data used for training and testing, respectively, may vary. In some cases, roughly 80% of the brain electrical signal data synchronized with movement data may be used for training and roughly 20% for testing. In some instances, roughly 75% of the synchronized data may be used for training and roughly 25% for testing.

In some embodiments, the training may further include validating the trained machine learning model or machine learning models. By validating in this context is meant evaluating the machine learning model during training using brain electrical signal data synchronized with movement data different from the brain electrical signal data synchronized with movement data used for training and testing. In some embodiments, a first subset of the synchronized data is used for training, a second subset of the synchronized data is used for testing, and a third subset of the synchronized data is used for validating. The validating may use one or more metrics to evaluate the performance of the machine learning model or machine learning models such as, e.g., any of the metrics discussed above for testing. In some embodiments, the validating may be used to, e.g., select model parameters (e.g., select one or more machine learning algorithms to continue training), optimize or tune hyperparameters (e.g., model hyperparameters or algorithm hyperparameters), etc. In some embodiments, the ensemble machine learning models were optimized using 10-fold cross-validation.

In some embodiments, trained machine learning models such as, e.g., trained models exhibiting above a threshold level of accuracy may be used to generate control signals for real time aDBS. In embodiments where the alternating bilateral movement includes walking, the adaptive deep brain stimulation may be used in order to improve gait function in patients with Parkinson's disease. In some embodiments, the control signals generated using the trained machine learning model(s) drive aDBS to improve gait function in PD patients by rapidly changing stimulation parameters in response to identified neural activity biomarkers of alternating bilateral movement in the subject.

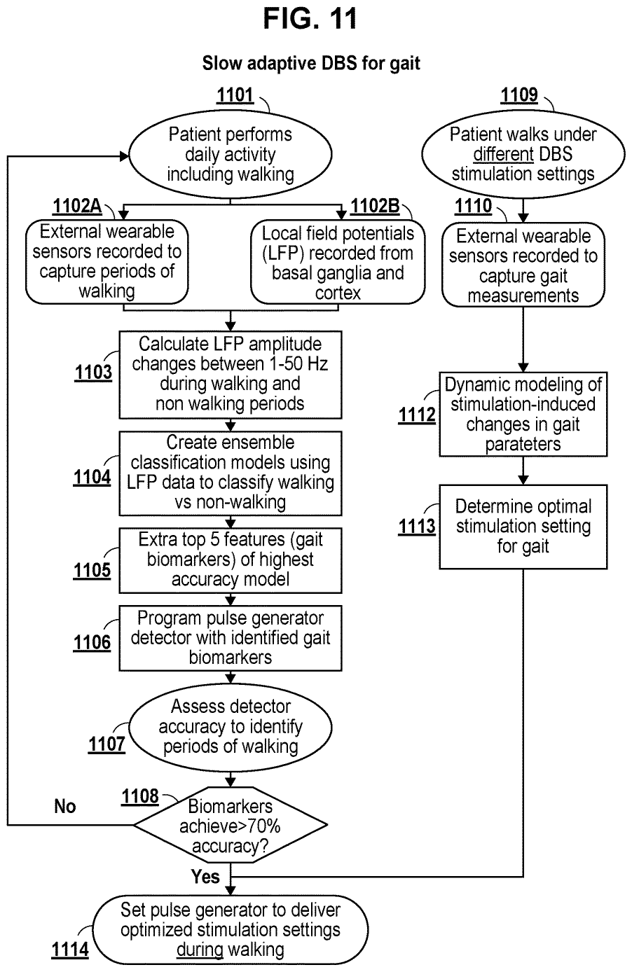

FIG. 11 provides a flow diagram depicting a method of slow adaptive DBS for gait in accordance with an embodiment of the invention. At step 1101, a patient performs a daily activity including an alternating bilateral movement such as walking. At step 1102A, a wearable sensor is used to record periods of walking and at step 1102B a neural recording device is used to record brain electrical signal data (i.e., local field potentials) as the subject walks. The movement data (i.e., recorded periods of walking) and the brain electrical signal data are then synchronized, e.g., by aligning acceleration peaks obtained from an accelerometer of the wearable sensor and acceleration peaks obtained from an accelerometer of the pulse generator used to transmit the brain electrical signal data to the processor. At step 1103, the brain electrical signal data synchronized with the movement data is then further processed to calculate amplitude (i.e., power) changes between 1 Hz and 50 Hz occurring during walking and non-walking periods. At step 1104, the processed data (e.g., from step 1103) is used to train ensemble classification models including, e.g., RF and LDA models, to classify periods of walking and non-walking from recorded brain electrical signal data. At step 1105, the top five gait biomarkers (i.e., identified by the classification models) are selected and used to program a pulse generator detector at step 1106. At step 1107, the programmed pulse generator detector is assessed on its ability to accurately classify periods of walking (step 1108). If the programmed pulse generator detector achieves an accuracy of below 70% for classifying/identifying periods of walking, steps 1101 through 1108 are repeated in order to generate new gait biomarkers. Once the programmed pulse generator detector achieves an accuracy of 70% or higher, the gait biomarkers are used to generate control signals for adaptive deep brain stimulation (step 1114). In other words, the programmed pulse generator detector detects brain electrical signal data (i.e., local field potentials) transmitted by the pulse generator, and uses the gait biomarkers to identify if the subject is walking. If the subject is determined to be walking, stimulation settings optimized for the subject during walking (e.g., in steps 1109 to 1113) are delivered. Optimized stimulation settings are determined by having the patient walk under different DBS stimulation settings (step 1109) while using the wearable sensor to capture gait measurements (step 1110). At step 1112, dynamic modeling of stimulation-induced changes in gait parameters is used to determined optimal stimulation settings for gait (step 1113).

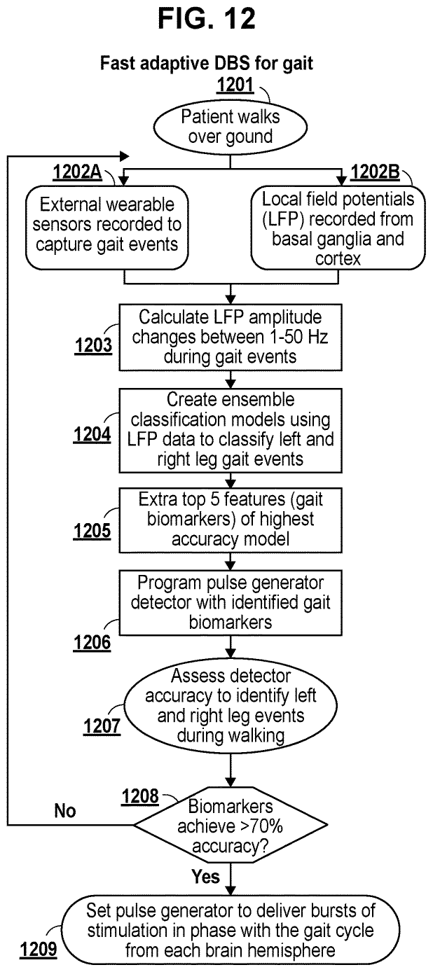

FIG. 12 provides a flow diagram depicting a method of fast adaptive DBS for gait in accordance with an embodiment of the invention. At step 1201, a patient performs an alternating bilateral movement such as walking. At step 1202A, a wearable sensor is used to record gait events (e.g., heel strikes, left stance phases, right swing phases, etc.) and at step 1202B a neural recording device is used to record brain electrical signal data (i.e., local field potentials) as the subject walks. The movement data (i.e., recorded gait events) and the brain electrical signal data are then synchronized, e.g., by aligning acceleration peaks obtained from an accelerometer of the wearable sensor and acceleration peaks obtained from an accelerometer of the pulse generator used to transmit the brain electrical signal data to the processor. At step 1203, the brain electrical signal data synchronized with the movement data is then further processed to calculate amplitude (i.e., power) changes between 1 Hz and 50 Hz occurring during gate events. At step 1204, the processed data (e.g., from step 1203) is used to train ensemble classification models including, e.g., RF and LDA models, to classify left and right leg gait events from recorded brain electrical signal data. At step 1205, the top five gait biomarkers (i.e., identified by the classification models) are selected and used to program a pulse generator detector at step 1206. At step 1207, the programmed pulse generator detector is assessed on its ability to accurately classify left and right leg events during walking (step 1208). If the programmed pulse generator detector achieves an accuracy of below 70% for classifying/identifying left and right leg events, steps 1201 through 1208 are repeated in order to generate new gait biomarkers. Once the programmed pulse generator detector achieves an accuracy of 70% or higher, the gait biomarkers are used to generate control signals for adaptive deep brain stimulation (step 1209). In other words, the programmed pulse generator detector detects brain electrical signal data (i.e., local field potentials) transmitted by the pulse generator, and uses the gait biomarkers to identify the phase of the gait cycle the patient is in. Bursts of stimulation are then appropriately delivered to each brain hemisphere in accordance with the identified phase of the gait cycle.

### Systems

Aspects of the present disclosure further include systems, such as computer-controlled systems, for practicing embodiments of the above methods. Aspects of the systems include: a neural recording device including an electrode adapted for positioning at a location in a sensorimotor cortex region or basal ganglia system of the brain of the subject to record brain electrical signal data associated with the subject performing the alternating bilateral movement; a sensor configured to record one or more body parts of the subject performing the movement; a processor configured to receive the brain electrical signal data from the neural recording device and the movement data from the sensor; and memory operably coupled to the processor wherein the memory includes instructions stored thereon, which when executed by the processor, cause the processor to process the brain electrical signal data and the movement data by synchronizing the brain electrical signal data and the movement data, and identify one or more neural activity biomarkers of the movement in the subject from the processed brain electrical signal data and movement data using a machine learning algorithm.

In some embodiments, the sensor is configured to record one or more of: the acceleration or velocity of the one or more body parts; the force generated by the one or more body parts; and the angle between the one or more body parts and a reference body part. In some embodiments, the sensor includes a wearable sensor. In some embodiments, the wearable sensor includes an accelerometer, a force sensitive resistor and/or a goniometer. In some embodiments, the sensor includes an image sensor and, e.g., the system further includes a motion tracking marker configured to be worn by the subject. In some cases, the sensor is a surface electromyography sensor.

In some embodiments, the neural recording device includes two or more electrodes. In some embodiments, one or more of the electrodes are adapted for positioning at a location in a sensorimotor cortex region of the brain and the basal ganglia system of the brain. In some cases, one or more of the electrodes are adapted for positioning at a location in both the left and right sensorimotor cortex region of the brain and/or both the left and right basal ganglia system of the brain. In some instances, the sensorimotor cortex region of the brain includes the precentral gyrus and/or the somatosensory cortex. In some cases, the basal ganglia system includes the subthalamic nucleus such as, e.g., the dorsal or ventral subthalamic nucleus. In other cases, the basal ganglia include the globus pallidum such as e.g., the globus pallidus internal or externa.

In some embodiments, the neural recording device includes a deep brain stimulation lead adapted for positioning in the basal ganglia system of the brain. In some embodiments, the neural recording device includes an electrocorticography (ECoG) paddle adapted for positioning in the sensorimotor cortex region of the brain. In some cases, both the deep brain stimulation lead adapted for positioning in the basal ganglia system of the brain and the electrocorticography (ECoG) paddle adapted for positioning in the sensorimotor cortex region of the brain are bilateral (e.g., are adapted for simultaneous positioning in both the left and right equivalents of the specific brain regions respectively).

In some embodiments, the system further includes an implantable pulse generator configured to wirelessly transmit the brain electrical signal data to the processor. In some cases, the implantable pulse generator includes an accelerometer used to synchronize the brain electrical signal data with the movement data.

In some embodiments, the processing further includes calculating one or more of a continuous wavelet transform (CWT), wavelet coherence, short-time Fourier transform (STFT), and power spectral density (PSD) using the synchronized brain electrical signal data and the movement data. In some cases, the machine learning algorithm includes a Random Forest (RF) algorithm.

In some embodiments, the memory includes instructions stored thereon, which when executed by the processor, cause the processor to train a machine learning model for identifying brain electrical signal data features of neural activity biomarkers using the processed brain electrical signal data and movement data and the RF algorithm. In other embodiments, this may include a recursive feature elimination algorithm. In some cases, the RF trained machine learning model is one of multiple ensemble models used for alternating bilateral movement event classification.

In some embodiments, the memory includes instructions stored thereon, which when executed by the processor, cause the processor to train a machine learning model of the ensemble of models for event classification using the processed brain electrical signal data and movement data and one or more of a K-nearest neighbors (KNN), logistic regression, linear discriminant analysis (LDA), Gradient Boosted Decision Trees (XGBoost), or neural network algorithm.

In some embodiments, the memory includes instructions stored thereon, which when executed by the processor, cause the processor to generate control signals for real time adaptive deep brain stimulation using recorded brain electrical signal data and the trained ensemble of machine learning models. In some cases, the memory includes instructions stored thereon, which when executed by the processor, cause the processor to generate control signals for real time adaptive deep brain stimulation using the identified neural activity biomarkers of alternating bilateral movement in the subject.

In some instances the systems further include one or more computers for complete automation or partial automation of the methods described herein. In some embodiments, systems include a computer having a computer readable storage medium with a computer program stored thereon.

In embodiments, the system includes an input module, a processing module and an output module. The subject systems may include both hardware and software components, where the hardware components may take the form of one or more platforms, e.g., in the form of servers, such that the functional elements, i.e., those elements of the system that carry out specific tasks (such as managing input and output of information, processing information, etc.) of the system may be carried out by the execution of software applications on and across the one or more computer platforms represented of the system.

Systems may include a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C++, Python, other high-level or low-level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques. The processor may be any suitable analog or digital system. In some embodiments, the processor includes analog electronics which provide feedback control, such as for example negative feedback control.

The system memory may be any of a variety of known or future memory storage devices. Examples include any commonly available random access memory (RAM), magnetic medium such as a resident hard disk or tape, an optical medium such as a read and write compact disc, flash memory devices, or other memory storage device. The memory storage device may be any of a variety of known or future devices, including a compact disk drive, a tape drive, a removable hard disk drive, or a diskette drive. Such types of memory storage devices typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with the memory storage device.

In some embodiments, a computer program product is described including a computer usable medium having control logic (computer software program, including program code) stored therein. The control logic, when executed by the processor the computer, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Memory may be any suitable device in which the processor can store and retrieve data, such as magnetic, optical, or solid-state storage devices (including magnetic or optical disks or tape or RAM, or any other suitable device, either fixed or portable). The processor may include a general-purpose digital microprocessor suitably programmed from a computer readable medium carrying necessary program code. Programming can be provided remotely to processor through a communication channel, or previously saved in a computer program product such as memory or some other portable or fixed computer readable storage medium using any of those devices in connection with memory. For example, a magnetic or optical disk may carry the programming, and can be read by a disk writer/reader. Systems of the invention also include programming, e.g., in the form of computer program products, algorithms for use in practicing the methods as described above. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; portable flash drive; and hybrids of these categories such as magnetic/optical storage media.

The processor may also have access to a communication channel to communicate with a user at a remote location. By remote location is meant the user is not directly in contact with the system and relays input information to an input manager from an external device, such as a computer connected to a Wide Area Network (“WAN”), telephone network, satellite network, or any other suitable communication channel, including a mobile telephone (i.e., smartphone).

In some embodiments, systems according to the present disclosure may be configured to include a communication interface. In some embodiments, the communication interface includes a receiver and/or transmitter for communicating with a network and/or another device. The communication interface can be configured for wired or wireless communication, including, but not limited to, radio frequency (RF) communication (e.g., Radio-Frequency Identification (RFID), Zigbee communication protocols, WiFi, infrared, wireless Universal Serial Bus (USB), Ultra Wide Band (UWB), Bluetooth® communication protocols, and cellular communication, such as code division multiple access (CDMA) or Global System for Mobile communications (GSM).

In one embodiment, the communication interface is configured to include one or more communication ports, e.g., physical ports or interfaces such as a USB port, an RS-232 port, or any other suitable electrical connection port to allow data communication between the subject systems and other external devices such as a computer terminal (for example, at a physician's office or in hospital environment) that is configured for similar complementary data communication.

In one embodiment, the communication interface is configured for infrared communication, Bluetooth® communication, or any other suitable wireless communication protocol to enable the subject systems to communicate with other devices such as computer terminals and/or networks, communication enabled mobile telephones, personal digital assistants, or any other communication devices which the user may use in conjunction.

In one embodiment, the communication interface is configured to provide a connection for data transfer utilizing Internet Protocol (IP) through a cell phone network, Short Message Service (SMS), wireless connection to a personal computer (PC) on a Local Area Network (LAN) which is connected to the internet, or WiFi connection to the internet at a WiFi hotspot.

In one embodiment, the subject systems are configured to wirelessly communicate with a server device via the communication interface, e.g., using a common standard such as 802.11 or Bluetooth® RF protocol, or an IrDA infrared protocol. The server device may be another portable device, such as a smart phone, Personal Digital Assistant (PDA) or notebook computer; or a larger device such as a desktop computer, appliance, etc. In some embodiments, the server device has a display, such as a liquid crystal display (LCD), as well as an input device, such as buttons, a keyboard, mouse or touch-screen.

In some embodiments, the communication interface is configured to automatically or semi-automatically communicate data stored in the subject systems, e.g., in an optional data storage unit, with a network or server device using one or more of the communication protocols and/or mechanisms described above.

Output controllers may include controllers for any of a variety of known display devices for presenting information to a user, whether a human or a machine, whether local or remote. If one of the display devices provides visual information, this information typically may be logically and/or physically organized as an array of picture elements. A graphical user interface (GUI) controller may include any of a variety of known or future software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g., over the Internet, phone or satellite network, in accordance with known techniques. The presentation of data by the output manager may be implemented in accordance with a variety of known techniques. As some examples, data may include SQL, HTML or XML documents, email or other files, or data in other forms. The data may include Internet URL addresses so that a user may retrieve additional SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a workstation, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows, iOS, Oracle Solaris, Linux, IBM, Unix, and others.

Aspects of the present disclosure further include non-transitory computer readable storage mediums having instructions for practicing the subject methods. Computer readable storage mediums may be employed on one or more computers for complete automation or partial automation of a system for practicing methods described herein. In certain embodiments, instructions in accordance with the method described herein can be coded onto a computer-readable medium in the form of “programming”, where the term “computer readable medium” as used herein refers to any non-transitory storage medium that participates in providing instructions and data to a computer for execution and processing. Examples of suitable non-transitory storage media include a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to the computer. A file containing information can be “stored” on computer readable medium, where “storing” means recording information such that it is accessible and retrievable at a later date by a computer. The computer-implemented method described herein can be executed using programming that can be written in one or more of any number of computer programming languages. Such languages include, for example, Python, Java, Java Script, C, C#, C++, Go, R, Swift, PHP, as well as many others.

The non-transitory computer readable storage medium may be employed on one or more computer systems having a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as those mentioned above, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques.

### Utility

The methods and systems of the invention find use in decoding alternating bilateral movement events directly from the neural activity of a subject. In particular, methods and systems are provided that allow for the identification of left and right leg events during walking from alternating power and coherence fluctuations between brain hemispheres of a subject. In some embodiments, the methods and systems described herein find use in regulating continuous bipedal locomotion in humans. Embodiments of the present disclosure find use in applications wherein it is desired to improve gait functions of patients with Parkinson's disease. In some embodiments, the subject methods and systems may be used to improve gait function in Parkinson's disease patients by decoding subject-specific gait biomarkers used to drive adaptive deep brain stimulation directly from the neural activity of a subject.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered 1-71 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of identifying neural activity biomarkers of an alternating bilateral movement in a subject, the method comprising:
positioning a neural recording device comprising one or more electrodes at a location in a sensorimotor cortex region or basal ganglia system of the brain of the subject to record brain electrical signal data associated with the subject performing the alternating bilateral movement;
instructing the subject to perform an activity comprising the alternating bilateral movement;
recording the brain electrical signal data associated with the movement performed by the subject using the neural recording device, wherein the brain electrical signal data is transmitted to a processor;
recording one or more body parts of the subject performing the movement using a sensor, wherein the movement data recorded by the sensor is transmitted to the processor;
processing the brain electrical signal data and the movement data using the processor, wherein the processing comprises synchronizing the brain electrical signal data and the movement data; and
identifying one or more neural activity biomarkers of the movement in the subject from the processed brain electrical signal data and movement data using the processor, wherein the processor is programmed to use a machine learning algorithm for the identification.

2. The method of aspect 1, wherein the subject has a neurological disorder affecting movement.

3. The method of aspect 2, wherein the neurological disorder is essential tremor, Parkinson's disease, and/or dystonia.

4. The method of aspect 3, wherein the neurological disorder is Parkinson's disease.

5. The method of aspect 4, wherein the subject is on medication to treat the Parkinson's disease.

6. The method of aspect 5, wherein the medication is a dopaminergic medication.

7. The method according to any of the preceding aspects, wherein movement is repeated 5 or more times.

8. The method according to aspect 7, wherein the movement is repeated 10 or more times.

9. The method according to aspects 7 or 8, wherein the movement comprises the repeated abduction, adduction, flexion, extension, and/or circumduction of one or more body parts of the subject.

10. The method according to aspect 9, wherein the movement comprises the repeated flexion and extension of one or more body parts of the subject.

11. The method according to aspect 10, wherein the one or more body parts comprises the subjects arms and/or legs.

12. The method according to aspect 11, wherein the movement comprises walking.

13. The method according to any of the preceding aspects, wherein the sensor is configured to record one or more of: the acceleration or velocity of the one or more body parts; the force generated by the one or more body parts; and the angle between the one or more body parts and a reference body part.

14. The method according to aspect 13, wherein the sensor comprises a wearable sensor.

15. The method according to aspect 14, wherein the wearable sensor comprises an accelerometer, a force sensitive resistor and/or a goniometer.

16. The method according to aspect 13, wherein the sensor comprises an image sensor.

17. The method according to aspect 16, wherein the subject wears a motion tracking marker.

18. The method according to any of the preceding aspects, wherein the sensor is comprises an electromyography sensor.

19. The method according to aspect 18, wherein the electromyography sensor is a surface electromyography sensor.

20. The method according to any of the preceding aspects, wherein the neural recording device comprises two or more electrodes.

21. The method according to aspect 20, wherein one or more of the electrodes are positioned at a location in a sensorimotor cortex region of the brain and the basal ganglia system of the brain.

22. The method according to aspects 20 or 21, wherein one or more of the electrodes are positioned at a location in both the left and right sensorimotor cortex region of the brain and/or both the left and right basal ganglia system of the brain.

23. The method according to any of aspects 20 to 22, wherein the sensorimotor cortex region of the brain comprises the precentral gyrus.

24. The method according to aspect 23, wherein the precentral gyrus comprises the hand knob area of the precentral gyrus.

25. The method according to any of aspects 20 to 24, wherein the sensorimotor cortex region of the brain comprises the somatosensory cortex.

26. The method according to any of aspects 20 to 25, wherein the basal ganglia system comprises the subthalamic nucleus.

27. The method according to any of the preceding aspects, wherein the neural recording device comprises a deep brain stimulation lead configured to be positioned in the basal ganglia system of the brain.

28. The method according to aspect 23, wherein the neural recording device comprises bilateral leads.

29. The method according to any of the preceding aspects, wherein the neural recording device comprises an electrocorticography (ECoG) paddle configured to be positioned in the sensorimotor cortex region of the brain.

30. The method according to aspect 29, wherein the neural recording device comprises bilateral paddles.

31. The method according to any of aspects 27 to 30, wherein the brain electrical signal data is wirelessly transmitted to the processor using an implantable pulse generator.

32. The method according to any of the preceding aspects, wherein the processing further comprises preprocessing the brain electrical signal data using a high-pass and/or low-pass filter.

33. The method according to any of the preceding aspects, wherein electrical signal data comprises neural oscillations in a range from 4 Hz to 30 Hz.

34. The method according to any of the preceding aspects, wherein the processing further comprises calculating one or more of a continuous wavelet transform (CWT), wavelet coherence, short-time Fourier transform (STFT), and power spectral density (PSD) using the synchronized brain electrical signal data and the movement data.

35. The method according to any of the preceding aspects, wherein the machine learning algorithm comprises a Random Forest (RF) algorithm.

36. The method according to aspect 35, wherein the processed brain electrical signal data and movement data and the RF algorithm are used to train a machine learning model for identifying brain electrical signal data features of neural activity biomarkers.

37. The method according to aspect 36, wherein the RF trained machine learning model is one of multiple ensemble models used for alternating bilateral movement event classification.

38. The method according to aspect 37, wherein the machine learning algorithm comprises one or more of a K-nearest neighbors (KNN), logistic regression, linear discriminant analysis (LDA), Gradient Boosted Decision Trees (XGBoost), and neural network algorithm for training a machine learning model of the ensemble of models for event classification.

39. The method according to aspects 37 or 38, wherein the trained ensemble of machine learning models is used to generate control signals for real time adaptive deep brain stimulation.

40. The method according to any of aspects 35 to 39, wherein the identified neural activity biomarkers of alternating bilateral movement are used to generate control signals for real time adaptive deep brain stimulation.

41. A system for identifying neural activity biomarkers of alternating bilateral movement in a subject configured to perform the method according to any of aspects 1 to 40.

42. A system for identifying neural activity biomarkers of alternating bilateral movement in a subject, the system comprising:
a neural recording device comprising an electrode adapted for positioning at a location in a sensorimotor cortex region or basal ganglia system of the brain of the subject to record brain electrical signal data associated with the subject performing the alternating bilateral movement;
a sensor configured to record one or more body parts of the subject performing the movement;
a processor configured to receive the brain electrical signal data from the neural recording device and the movement data from the sensor; and
memory operably coupled to the processor wherein the memory comprises instructions stored thereon, which when executed by the processor, cause the processor to process the brain electrical signal data and the movement data by synchronizing the brain electrical signal data and the movement data, and identify one or more neural activity biomarkers of the movement in the subject from the processed brain electrical signal data and movement data using a machine learning algorithm.

43. The system according to aspect 42, wherein the sensor is configured to record one or more of: the acceleration or velocity of the one or more body parts; the force generated by the one or more body parts; and the angle between the one or more body parts and a reference body part.

44. The system according to aspect 43, wherein the sensor comprises a wearable sensor.

45. The system according to aspect 44, wherein the wearable sensor comprises an accelerometer, a force sensitive resistor and/or a goniometer.

46. The system according to aspects 42 or 43, wherein the sensor comprises an image sensor.

47. The system according to aspect 46, wherein the subject wears a motion tracking marker.

48. The system according to any of the aspects 42 to 44, wherein the sensor is comprises an electromyography sensor.

49. The system according to aspect 48, wherein the electromyography sensor is a surface electromyography sensor.

50. The system according to any of aspects 42 to 49, wherein the neural recording device comprises two or more electrodes.

51. The system according to aspect 50, wherein one or more of the electrodes are adapted for positioning at a location in a sensorimotor cortex region of the brain and the basal ganglia system of the brain.

52. The system according to aspects 50 or 51, wherein one or more of the electrodes are adapted for positioning at a location in both the left and right sensorimotor cortex region of the brain and/or both the left and right basal ganglia system of the brain.

53. The system according to any of aspects 50 to 52, wherein the sensorimotor cortex region of the brain comprises the precentral gyrus.

54. The system according to aspect 53, wherein the precentral gyrus comprises the hand knob area of the precentral gyrus.

55. The system according to any of aspects 50 to 54, wherein the sensorimotor cortex region of the brain comprises the somatosensory cortex.

56. The system according to any of aspects 50 to 55, wherein the basal ganglia system comprises the subthalamic nucleus.

57. The system according to any of aspects 42 to 56, wherein the neural recording device comprises a deep brain stimulation lead adapted for positioning in the basal ganglia system of the brain.

58. The system according to aspect 57, wherein the neural recording device comprises bilateral leads.

59. The system according to any of aspects 42 to 58, wherein the neural recording device comprises an electrocorticography (ECoG) paddle adapted for positioning in the sensorimotor cortex region of the brain.

60. The system according to aspect 59, wherein the neural recording device comprises bilateral paddles.

61. The system according to any of aspects 42 to 60, wherein the system further comprises an implantable pulse generator configured to wirelessly transmit the brain electrical signal data to the processor.

62. The system according to any of aspects 42 to 61, wherein the processing further comprises preprocessing the brain electrical signal data using a high-pass and/or low-pass filter.

63. The system according to any of aspects 42 to 62, wherein the electrical signal data comprises neural oscillations in a range from 4 Hz to 30 Hz.

64. The system according to any of aspects 42 to 63, wherein the processing further comprises calculating one or more of a continuous wavelet transform (CWT), wavelet coherence, short-time Fourier transform (STFT), and power spectral density (PSD) using the synchronized brain electrical signal data and the movement data.

65. The system according to any of aspects 42 to 64, wherein the machine learning algorithm comprises a Random Forest (RF) algorithm.

66. The system according to aspect 65, wherein the memory comprises instructions stored thereon, which when executed by the processor, cause the processor to train a machine learning model for identifying brain electrical signal data features of neural activity biomarkers using the processed brain electrical signal data and movement data and the RF algorithm.

67. The system according to aspect 66, wherein the RF trained machine learning model is one of multiple ensemble models used for alternating bilateral movement event classification.

68. The system according to aspect 67, wherein the memory comprises instructions stored thereon, which when executed by the processor, cause the processor to train a machine learning model of the ensemble of models for event classification using the processed brain electrical signal data and movement data and one or more of a K-nearest neighbors (KNN), logistic regression, linear discriminant analysis (LDA), Gradient Boosted Decision Trees (XGBoost), and neural network algorithm.

69. The system according to aspects 67 or 68, wherein the memory comprises instructions stored thereon, which when executed by the processor, cause the processor to generate control signals for real time adaptive deep brain stimulation using recorded brain electrical signal data and the trained ensemble of machine learning models.

70. The system according to any of aspects 65 to 69, wherein the memory comprises instructions stored thereon, which when executed by the processor, cause the processor to generate control signals for real time adaptive deep brain stimulation using the identified neural activity biomarkers of alternating bilateral movement in the subject.

71. A kit comprising the system of any of aspects 42 to 70 and instructions for identifying neural activity biomarkers of alternating bilateral movement in a subject.

### EXAMPLES

As demonstrated in the above disclosure, the present invention has a wide variety of applications. The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Efforts have been made to ensure accuracy with respect to numbers used (e.g. amounts, volumes, etc.) but some experimental errors and deviations should be accounted for.

### Overview

Human's ability to coordinate stereotyped, alternating movements between the two legs during bipedal walking is a complex motor behavior that requires precise timing activities across multiple nodes of the supraspinal network. Understanding of the neural network dynamics that underlie natural walking in humans is limited. Cortical and subthalamic neural activities were investigated during overground walking and spectral biomarkers were evaluated to decode the gait cycle in three patients with Parkinson's disease without gait disturbances. Patients were implanted with chronic bilateral deep brain stimulation leads in the subthalamic nucleus (STN) and electrocorticography paddles overlaying the primary motor (M1) and somatosensory (S1) cortices. Local field potentials (LFP) were recorded from these areas while the participants performed overground walking and synchronized to external gait kinematic sensors. It was found that the STN displays increased low frequency (4-12 Hz) spectral power during the period prior to contralateral leg swing. Furthermore, STN shows increased theta frequency (4-8 Hz) coherence with the primary motor through the initiation and early phase of contralateral leg swing. Additional analysis revealed that each patient had specific frequency bands which could detect a significant difference between left and right initial leg-swing. These findings indicate that there are alternating spectral changes between the two hemispheres in accordance with the gait cycle. In addition, patient-specific, gait-related biomarkers were identified in both the STN and cortical areas at discrete frequency bands that may be used to drive adaptive DBS to improve gait dysfunction in patients with Parkinson's disease.

By recording from chronically implanted electrodes from the subthalamic nucleus and sensorimotor cortex in patients with Parkinson's disease, power modulations were found across multiple frequency bands (4-30 Hz) during specific phases of the gait cycle. The coherence between subthalamic-cortical areas of each brain hemisphere also increases prior to contralateral leg swing. The data supports the hypothesis that the basal ganglia and cortex coordinate alternating power and coherence fluctuations between hemispheres, which indicates a mechanism to regulate continuous bipedal locomotion in humans. Lastly, it is shown that these putative biomarkers for gait can decode left and right gait events, implicating a potential use to drive future adaptive DBS algorithms.

### INTRODUCTION

The hypothesis that the STN interacts with the motor cortex in a temporal-specific manner to coordinate reciprocal leg movements to generate effective bipedal locomotion was explored by the experiments below. The cortical-subthalamic circuit dynamics of natural walking were investigated from three patients with PD without major gait disturbances in the on-mediation state to capture the most physiological gait possible. Patients were implanted with chronic bilateral STN DBS leads and sensorimotor cortex electrocorticography (ECoG) paddles. Neural oscillatory activities were simultaneously and wirelessly streamed (through implanted bidirectional pulse generators) from the bilateral primary motor (M1) and somatosensory (S1) cortices as well as the STN during overground walking and were synchronized to external gait kinematic sensors. The aims were: 1) to characterize the oscillatory signatures of natural walking from the STN and sensorimotor cortices, 2) to identify cortico-subthalamic circuit coherence changes throughout the gait cycle, and 3) to determine accuracy of gait event decoding (i.e., heel-strike or toe-off) based on these cortical and subthalamic oscillatory signatures.

### Materials and Methods

### Subjects and Electrode Reconstruction

Three male subjects with idiopathic PD undergoing evaluation for DBS surgery were enrolled at the University of California-San Francisco. Subjects did not exhibit major gait impairments, with MDS-UPDRS III postural instability and gait sub-scores on medication between 1 (slight) to 2 (mild) (Table 1). All subjects provided written informed consent (NCT03582891).

TABLE 1
Subject Demographics

| | Age/ | Disease | DBS | UPDRS III Total | UPDRS III Total | UPDRS III PIGD* |
|---|---|---|---|---|---|---|
| ID | Sex | Duration | Target | Off-meds | On-meds | On-meds |
| Subject 1 | 42/M | 06 | STN | 41 | 14 | 2 |
| Subject 2 | 58/M | 09 | STN | 34 | 09 | 1 |
| Subject 3 | 61/M | 05 | STN | 35 | 12 | 1 |

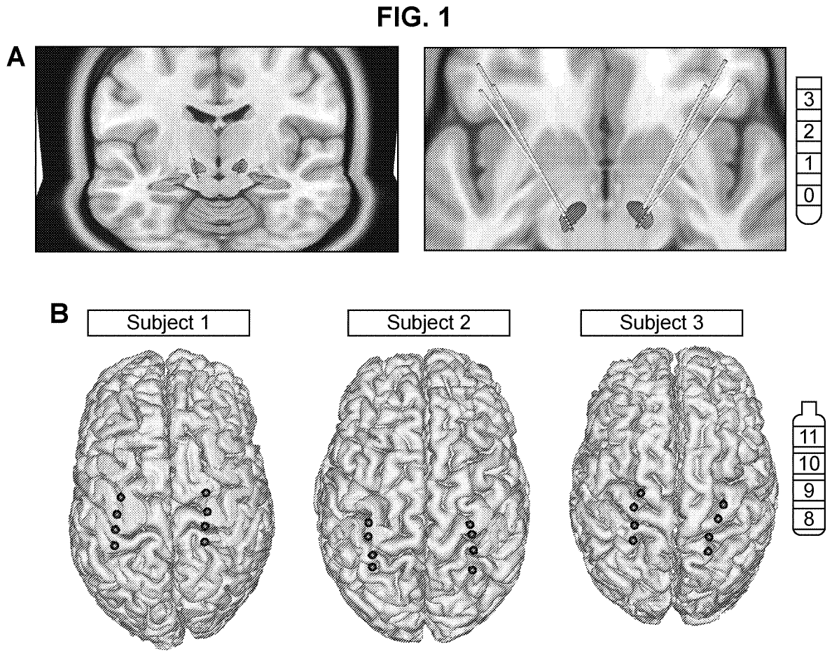

All subjects underwent bilateral implantation of quadripolar DBS leads into the STN (Medtronic model 3389), quadripolar cortical paddle overlying the sensorimotor cortices (Medtronic model 0913025), connected to bilateral investigational sensing pulse generators (Medtronic Summit RC+S model B35300R) as previously described (FIG. 1) [1]. Each RC+S device was connected to an STN DBS electrode and a cortical paddle from the same brain hemisphere.

DBS and cortical electrode localization were performed using 2-month postoperative CT images fused with preoperative T1-weighted MRI images. STN DBS lead reconstruction was performed using the DISTAL atlas and TRAC/CORE algorithm available within LEAD-DBS, an open-source MATLAB toolbox [2] [3]. Intracranial EEG Anatomical Processing and Electrode Reconstruction Pipeline (https://edden-gerber.github.io/ecog_recon/) was used for cortical paddle reconstruction. T1 images were parcellated and converted into a standardized cortical surface mesh using FreeSurfer [4] and AFNI's SUMA [5]. Cortical contacts were then manually identified on the CT images in Biolmage Suite [6] and the electrode coordinates were projected onto the standardized mesh using a gradient descent algorithm in MATLAB.

### Neural Recordings and Gait Kinematic Measurements During Natural Walking

Subjects walked overground at their preferred speed for 2 minutes in a straight path of at least 15 feet before turning around. All subjects were on their typical dose of Parkinsonian medication during the task. In all subjects LFPs were recorded from two STN electrode pairs: ventral STN (+2-0) and dorsal STN (+3-1), where contact 0 is in the ventral STN, contact 3 just above the dorsal border, and contacts 1 and 2 in the motor territory based on microelectrode mapping (FIG. 1A). The two cortical electrode recording configuration were +9-8 (S1) and +11-10 (M1), based on and imaging reconstruction. LFPs were sampled at 500 Hz and passed through a pre-amplifier high-pass filter of 0.85 Hz and low-pass filter of 450 Hz. Accelerometry data from the Summit RC+S system was sampled at 64 Hz. All data from the RC+S system was extracted and analyzed using open-source code (https://github.com/openmind-consortium/Analysis-rcs-data).

Gait kinematic data was collected using two wireless sensor systems: Delsys Trigno® system (Delsys Inc. Natick, MA) and Xsens MVN Analyze (Xsens Technologies, The Netherlands). The Delsys sensors included two Avanti force sensitive resistor (FSR) adapters, two Avanti goniometer adapters, and two Trigno surface electromyography (EMG) sensors with a built-in accelerometer. The Avanti adapters were placed bilaterally on the shank of the leg, and the EMG sensors were placed on top of both RC+S and used for synchronization (see below). Each FSR adapter was attached to four FSRs (Delsys DC: F01) placed under the calcaneus, hallux, 1st metatarsal (1MT), and 5th metatarsal (5MT). Digital goniometer (SG110/A) was placed next to the lateral malleolus. The Xsens system is comprised of 14 inertial measurement unit sensors placed over the entire body and limbs for wireless motion tracking.

FIGS. 1A to 1B: DBS and cortical lead localization. (A) 3D reconstructions of all DBS lead locations in the STN (orange). Individual subject's leads are shown in different colors. (B) 3D reconstructions of cortical electrode paddle location. The two most anterior contacts overlie the primary motor cortex (M1), while the two most posterior contacts overlie the somatosensory cortex (S1).

### Data Analysis

LFP and gait kinematic data was synchronized by aligning the acceleration peaks captured by the RC+S, Delsys Trigno sensors over the RC+S, and Xsens accelerometry. Four signal processing methods were applied to the LFP signals using built in MATLAB functions: continuous wavelet transform (CWT; “cwt” function), wavelet coherence (“wcoherence” function), short-time Fourier transform (STFT; “spectrogram” function), and power spectral density (PSD; “spectrogram” function with 1 second window, 90% window overlap, and a transform length of 512 data points). Wavelet transformation was used because it has greater low-frequency resolution. The Fourier transform was also used because this is the on-board spectral decomposition method used by the RC+S system [7].

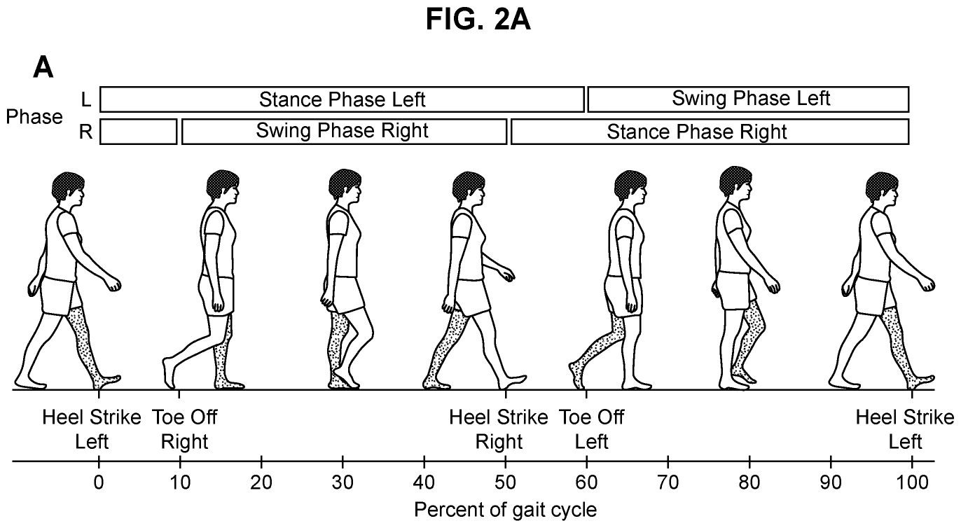

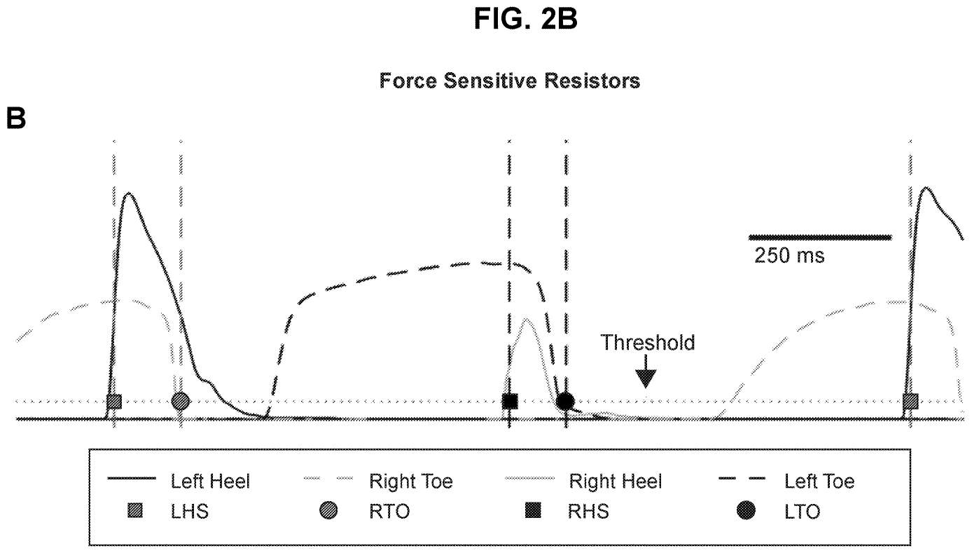

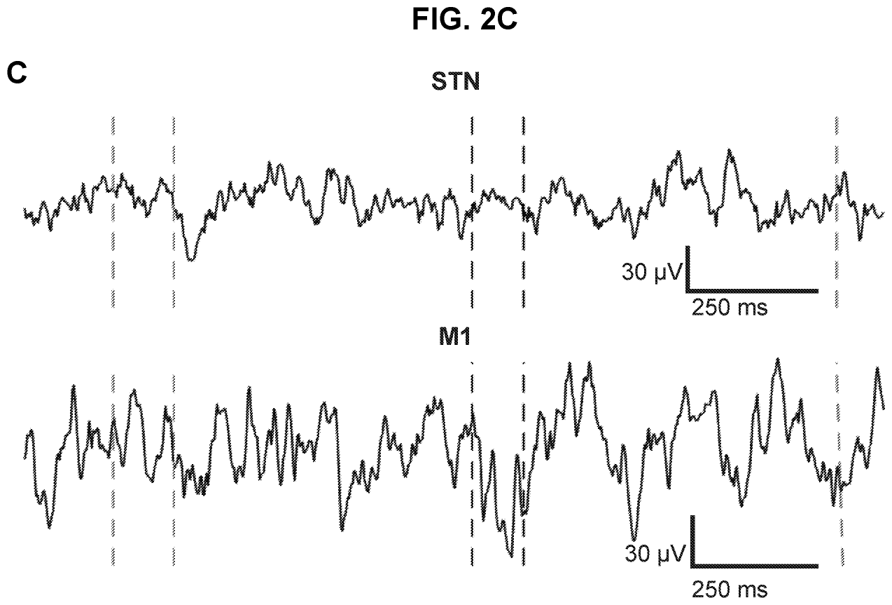

Gait kinematic data was used to determine left and right toe-off and heel-strike events using a custom MATLAB script (FIG. 2). Heel-strike was defined as the time when the calcaneus or 5MT FSR crosses over a 5% threshold in the positive direction. Toe-off was defined as the time when the hallux or 1MT FSR crosses over the 5% threshold in the negative direction. For the Xsens system, toe-off was defined as the time of peak ankle plantarflexion velocity, while heel-strike was defined as the time of ankle velocity impulse. All gait events were visually inspected, and erroneous events were manually corrected. Turns were excluded from analysis. 40 gait cycles were included for analysis from subject 1, 67 for subject 2, and 106 for subject 3.

Individual gait cycle epochs were extracted from the CWT and wavelet coherence data and divided into time bins representing 1% of the gait cycle. Power and magnitude-square coherence values for each gait cycle were normalized to the average value during the entire walking period by z-score. Z-scored values for gait cycles were then averaged across subjects to obtain the grand average spectrogram and coherogram.

To identify frequency bands where power differed between gait events, instantaneous power at each gait event (left and right toe-off and heel-strike) were extracted. All possible frequency bands were created between 0-50 Hz, and a Kruskal-Wallis test was used to identify frequency bands where power differed among the gait events. A Kruskal-Wallis test was used because the data sets were not normally distributed (Shapiro-Wilk test), but the variances of the different gait events were equal (Levene's test). P-values were adjusted using Tukey's Honest Significant Difference method. Frequency bands where the multiple comparison test reached p-values <0.05 were designated as gait-event-modulated frequency bands.

FIGS. 2A to 2C: Synchronized gait kinematic data with raw local field potential recordings during natural walking. (A) Illustration of gait events and phases during a single gait cycle, aligned to left heel-strike (0% gait cycle). (B) Heel-strike (squares) and toe-off (circles) gait events were detected from the left (black) and right (gray) force sensitive resistor data. Heel-strikes were detected when the heel force (solid line) exceeded a threshold (dotted line), and toe-offs were detected when toe force (dashed line) fell below the threshold. (C) Example local field potential recordings from both STN and M1 synchronized to a gait cycle.

### Gait Event Classification

A classification model was built to predict gait events from LFP power and the STN-cortical coherence. The classification model used an ensemble learning approach to enhance the stability and accuracy [8] [9] and consisted of a Random Forest (RF) feature selection model and a linear discriminant analysis (LDA) model. RF has been shown to achieve better performance than other feature selection methods [10], and is robust to collinearity [11]. The LDA model matched the on-board hardware classifier of the RC+S. The classifier models were built in R with the “Tidymodel” framework and trained for each subject, brain hemisphere, and recording area.

Features used in the RF model were instantaneous power or magnitude-squared coherence during toe-off events in all possible frequency bands between 2.5-50 Hz. All features were normalized to a mean of 0 and a standard deviation of 1. Prior to feature selection, RF hyperparameters, the number of decision tress and number of features a tree considers during node splitting, were optimized using 10-fold cross-validation with each data set stratified by toe-off classes. Once optimized, the RF feature selection model was trained on all normalized features using the “ranger” package in RStudio (www.rstudio.com).

The top ten features with the largest variable importance value based on “permutation importance” were used to generate new data sets for each subject and brain hemisphere. Next, the new data sets were split into 75% for training and 25% for testing. The accuracy and receiver operator characteristic area under the curve (AUC) were calculated.

### Statistical Analysis

Linear repeated-measure mixed model was used to determine power or coherence values that differed from the average during the gait cycle. A single fixed effect was used, and subjects were added to the model as a random to account for individual baseline neural power differences. Significance was tested using F-tests with Satterthwaite's degrees of freedom method. Statistical analysis of classification models were performed only on models that achieved greater than chance accuracy (≥50%). Significance was tested by permuting the toe-off class labels 1000 times and calculating the class accuracy on the permuted data. Models were determined to be significant if it correctly classified the event in <5% of total number of permutations [15].

### Example 1: STN Shows Coordinated Low Frequency Power Modulation During Walking

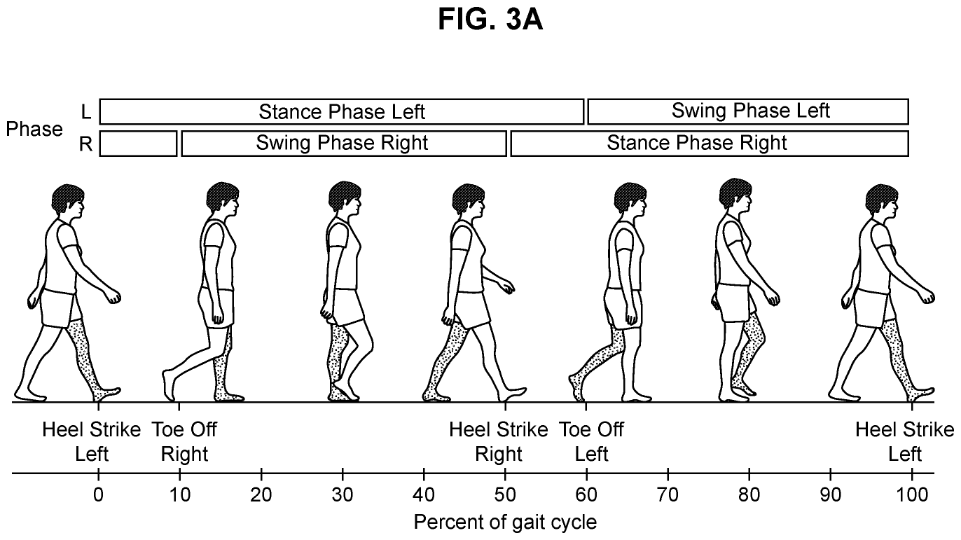

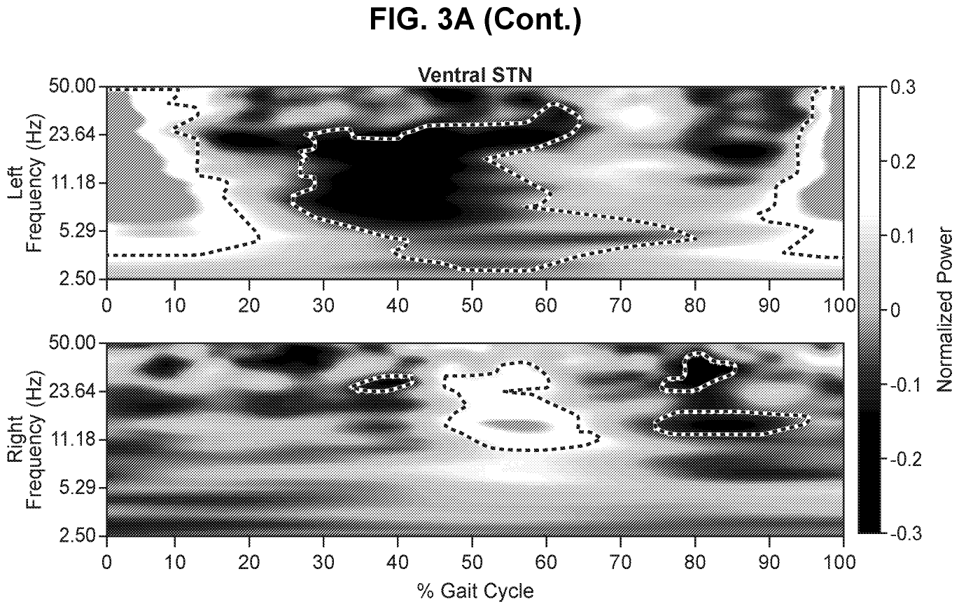

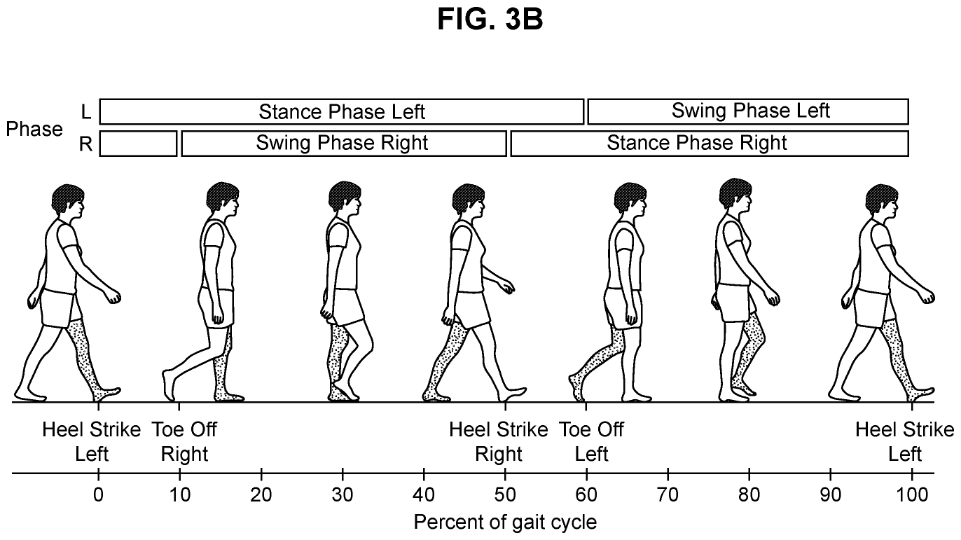

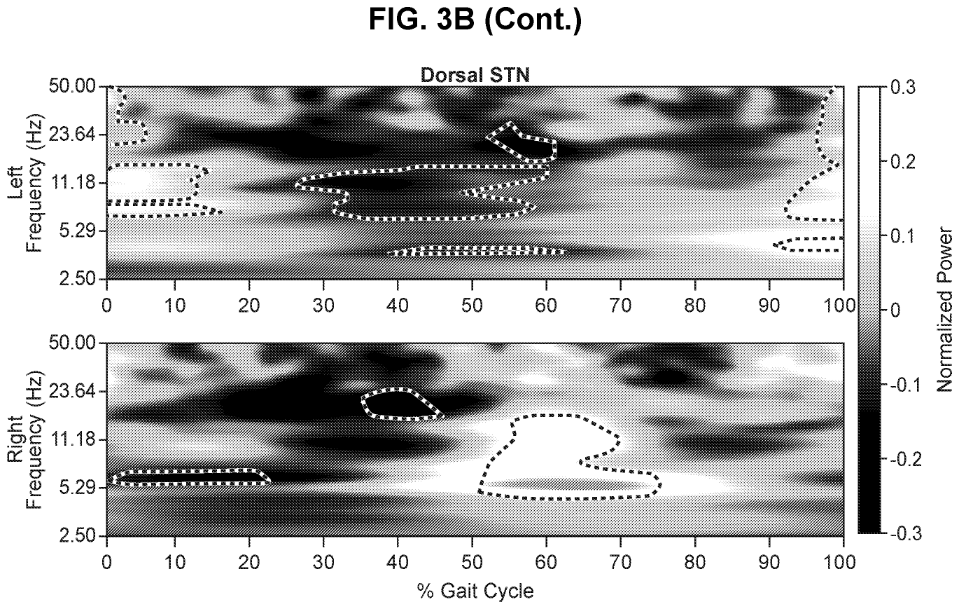

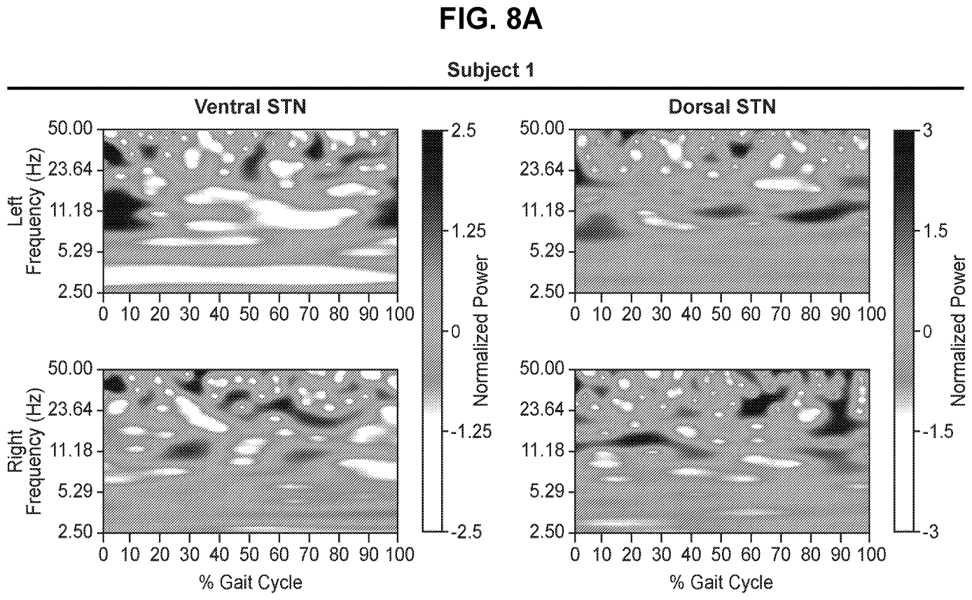

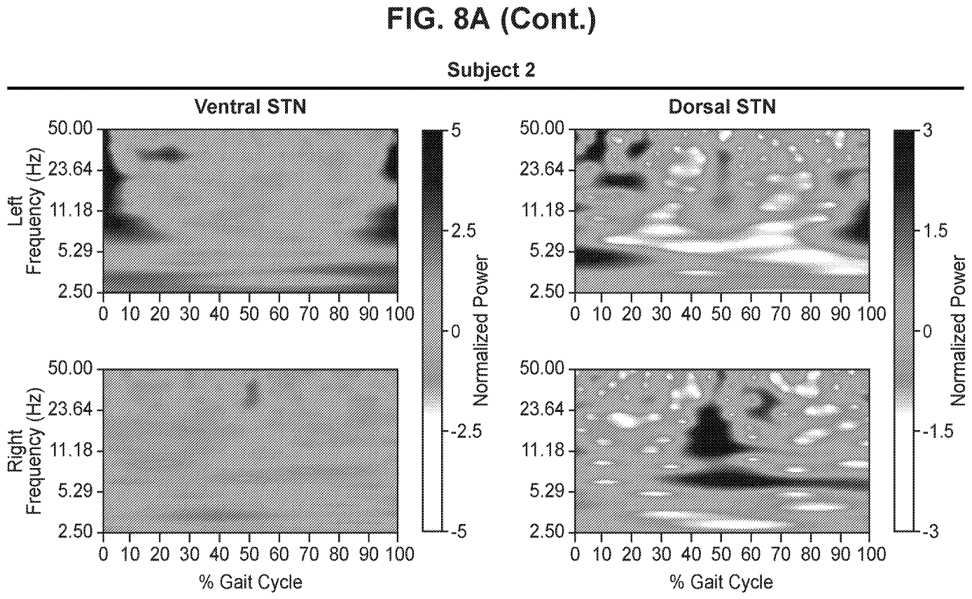

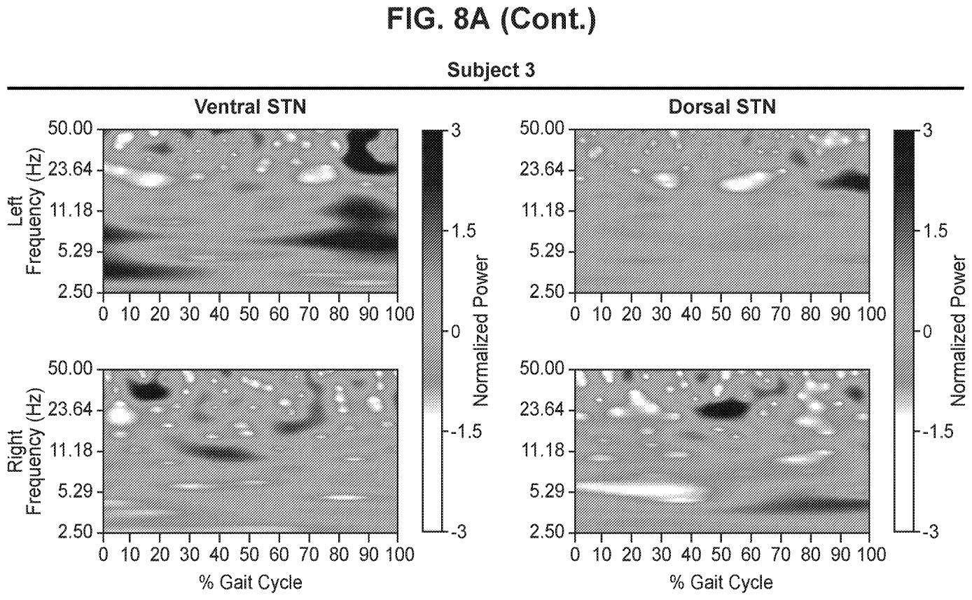

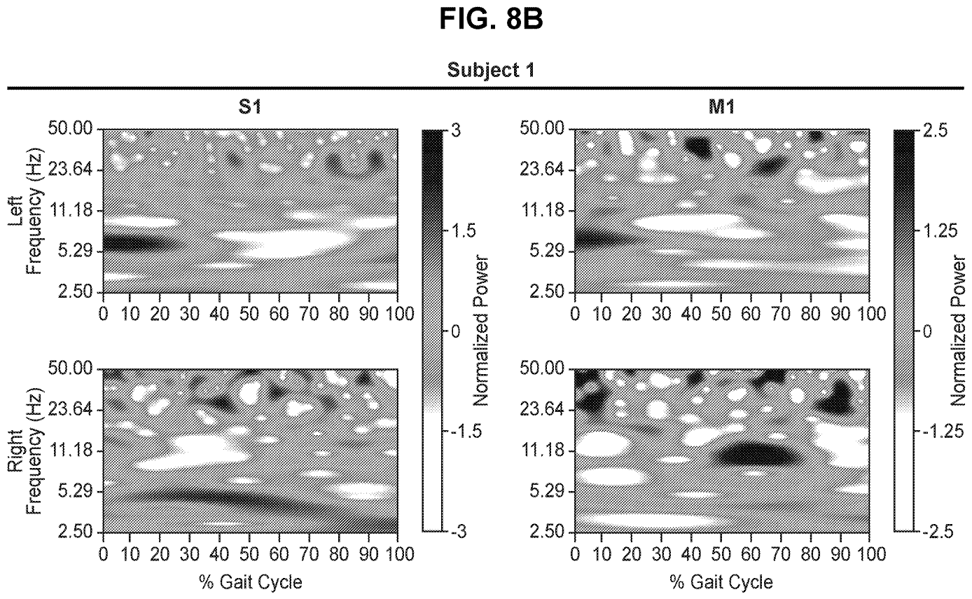

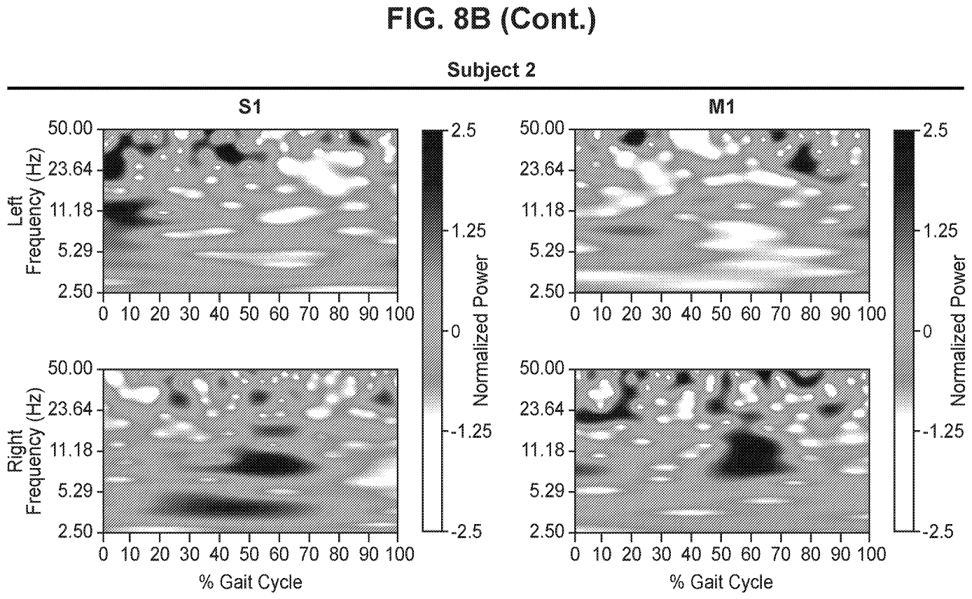

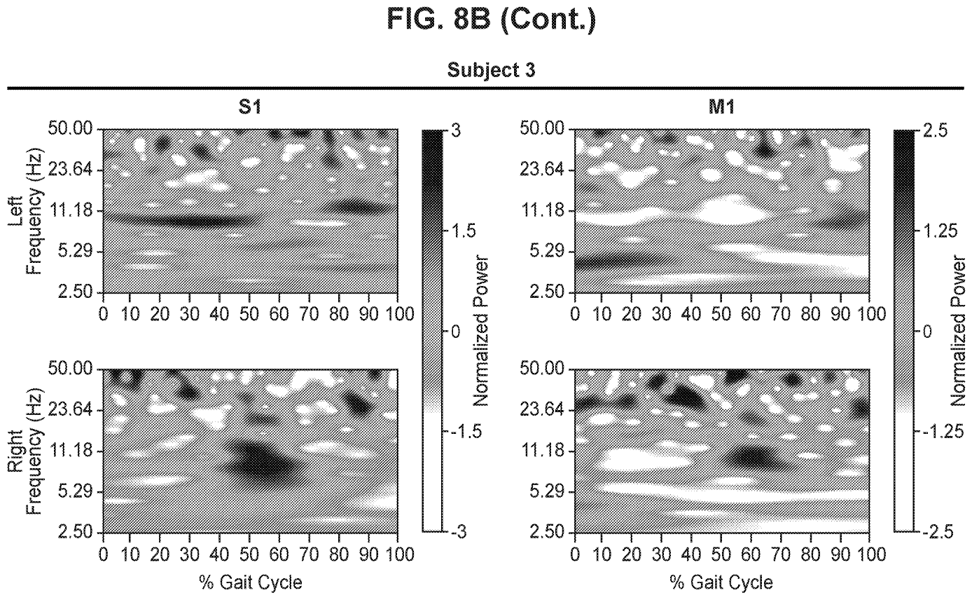

To investigate STN and sensorimotor cortical neural dynamics during the gait cycle, spectral power was extracted and averaged across all gait cycle epochs, and it was tested whether the power significantly changes during the gait cycle. It was found that the two hemispheres showed coordinated and reciprocal changes in spectral power within the ventral and dorsal STN during the gait cycle. Significant changes in power were seen in the alpha to low-gamma frequency (10-50 Hz) band power in the ventral STN, and in low frequency (5-15 Hz) band power in the dorsal STN. Increased power occurred during double support phase, the period from ipsilateral heel-strike to contralateral toe-off (0-10% for the left leg and 50-60% for the right leg) (FIGS. 3A and 3B, top). The left STN also demonstrated significant alpha-beta (8-30 Hz) power decrease during right leg swing period, and beta band (13-30 Hz) decrease during right heel-strike (FIGS. 3A and 3B, top). These changes in LFP power were also seen in individual gait cycles across all subjects (FIGS. 8A and 8B).

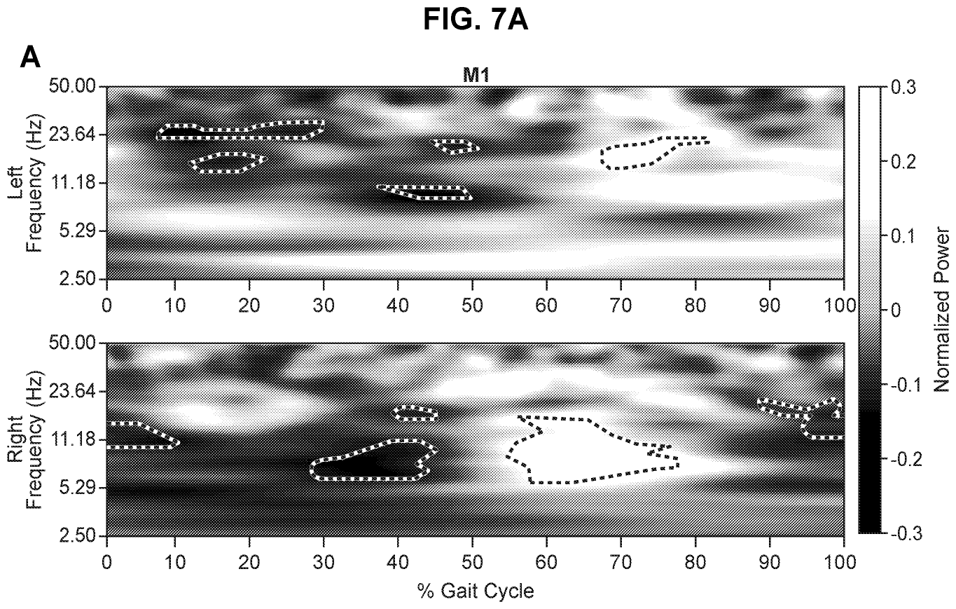

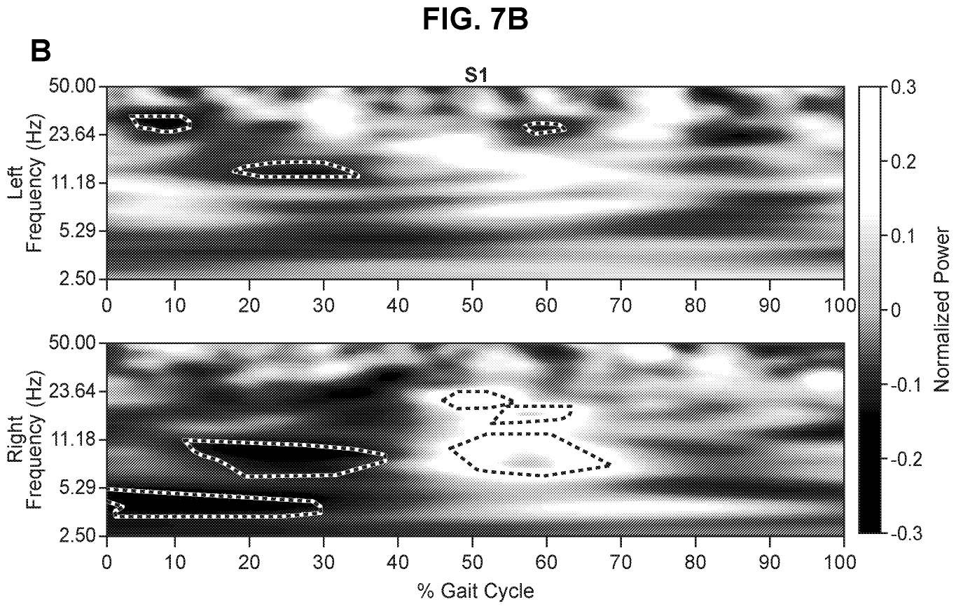

M1 and S1 also demonstrated power changes throughout the gait cycle, though the frequency-specific changes between the left and right hemispheres were not reciprocal. The left M1 showed decreased beta activity during right leg swing (10-30% of gait cycle) and increased beta power during right leg stance (60-80% of gait cycle) (FIG. 7A, top). While the right M1 does not show significant beta power modulation, it showed theta power changes during the end of right leg swing and beginning of left leg swing (FIG. 7A, bottom). The right S1 shows a similar pattern of theta modulation during transition from right leg swing to left leg swing (FIG. 7B, bottom).

FIGS. 3A to 3B: STN local field potentials show spectral power modulations during the gait cycle. Grand average z-score spectrograms from the dorsal and ventral STNs normalized to a gait cycle. (A-B) Significant power increases are seen during weight acceptance of the left leg in the left hemisphere (~0-10% gait cycle) and right leg in the right hemisphere (~50-60% gait cycle). Power increases were observed in a wide frequency band (10-50 Hz) in the ventral STN and in low frequency band (5-15 Hz) in the dorsal STN. Significant beta (13-30 Hz) desynchronization was also seen during contralateral leg swing and heel-strikes. (A-B) Gait cycle percentages and frequencies where power was significantly different compared to the average power during the entire walking task is outlined by the dashed white lines. A linear mixed-effect model was used to determine significance with p-value <0.05. FIG. 7 shows grand average gait cycles from cortical recorded contacts. FIGS. 8A to 8B show a single gait cycle from all recorded areas from all subjects in the study and show alternating left-right power changes throughout the gait cycle.

FIGS. 7A to 7B: Cortical local field potentials show spectral power modulations during the gait cycle. (A) Left M1 shows alpha (8-10 Hz) and beta desynchronization during right leg heels strike and initial right leg swing, respectively. Right M1 shows increased theta-alpha (5-12 Hz) during initial left leg swing and decreased beta around left heel-strike. (B) Significant decreased beta power is seen during left leg weight acceptance and initial right leg swing. Increases in theta-beta power (5-23 Hz) were seen during weight acceptance of the right leg and initial left leg swing.

FIGS. 8A to 8B: Spectrograms of a single gait cycle from the STN and sensorimotor cortices. All subjects show alternating left and right spectral power changes throughout the gait cycle.

### Example 2: STN Interacts with Motor and Sensory Cortices During Different Phases of the Gait Cycle

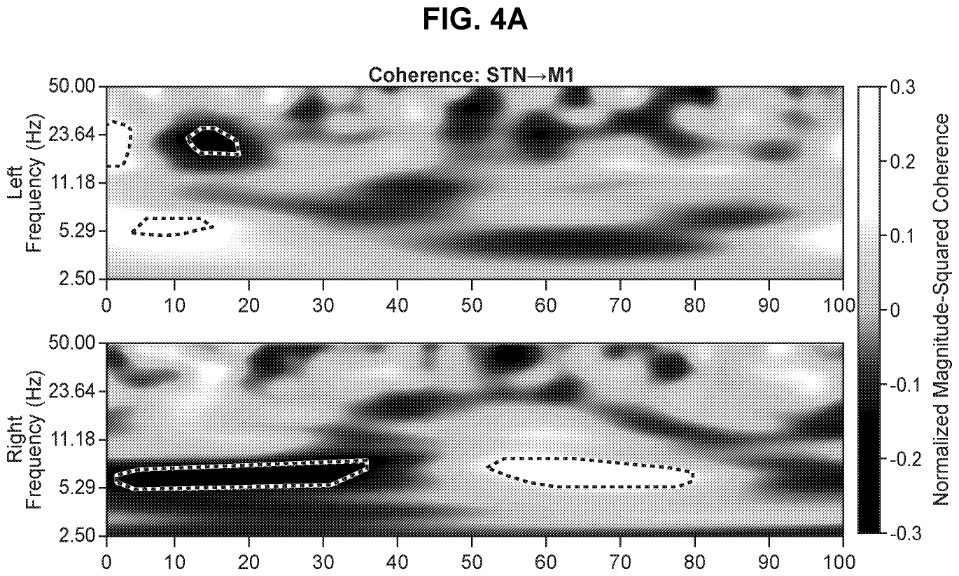

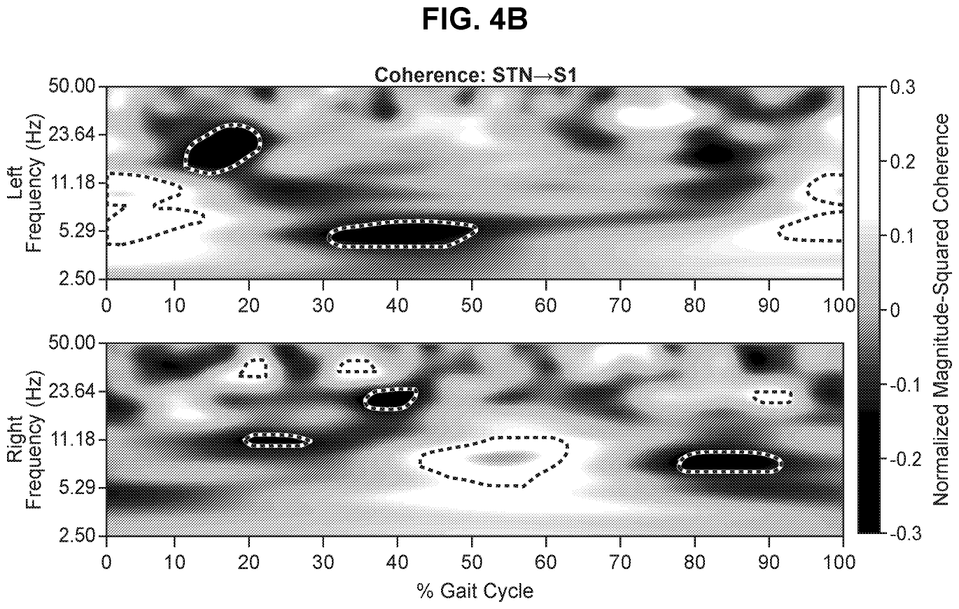

Because the STN has direct connections with sensorimotor cortices and plays important functions in motor control, it was examined whether the STN interacts with the cortex during specific phases of the gait cycle. To determine the nature and degree of this interaction, the averaged magnitude-squared coherence value was compared between the STN and M1/S1 for each brain hemisphere during the gait cycle. Increased STN-M1 theta band coherence was found during contralateral toe-off and initial contralateral leg swing, similar to the power modulations seen in the STN (FIG. 4A). Interesting, STN-S1 showed greater theta and alpha band coherence during ipsilateral heel-strike (FIG. 4B). The two brain hemispheres showed reciprocal coherence modulations.

FIGS. 4A to 4B: Low frequency STN/Cortical coherence increase during the initiation of contralateral leg swing. Grand average z-score coherogram from STN-M1 and -S1 normalized to a gait cycle. Reciprocal coherence modulation was seen in both hemispheres. (A) STN-M1 coherence showed significant increases in the theta band (5-8 Hz) during the initiation of contralateral leg swing through mid-swing. Additionally, the left hemisphere showed beta band coherence increases during initial ipsilateral weight acceptance. (B) STN-S1 coherence modulation was seen theta/alpha band across both hemispheres during ipsilateral heel-strike. (A-B) Gait cycle percentages and frequencies where coherence was significantly different from the average coherence during the entire walking task are outlined by the dashed white lines. A linear mixed-effect model was used to determine significance with p-value <0.05.

### Example 3: Patient-Specific Oscillatory Biomarkers of Gait

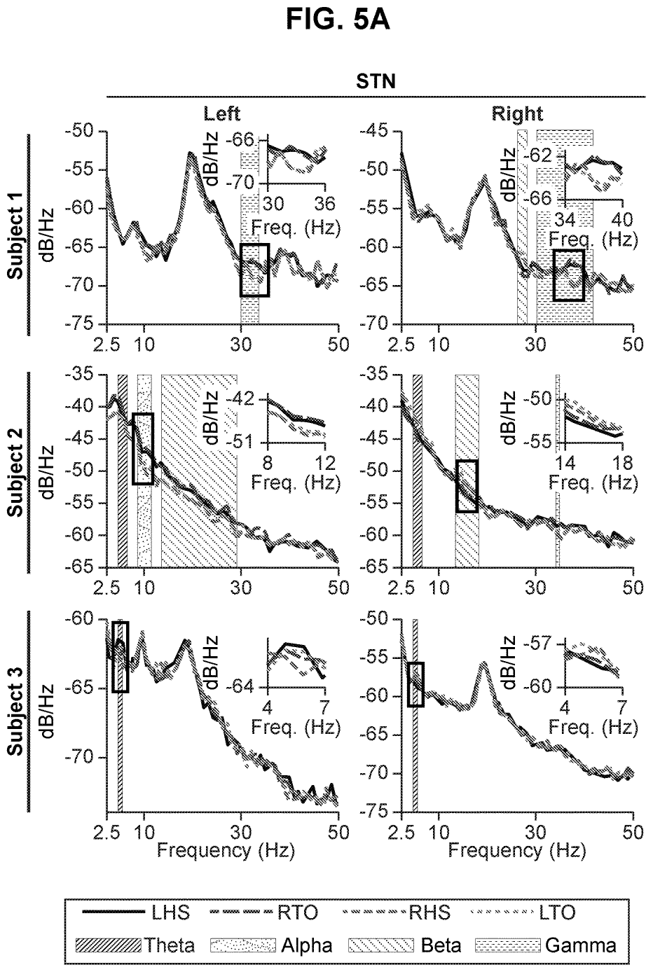

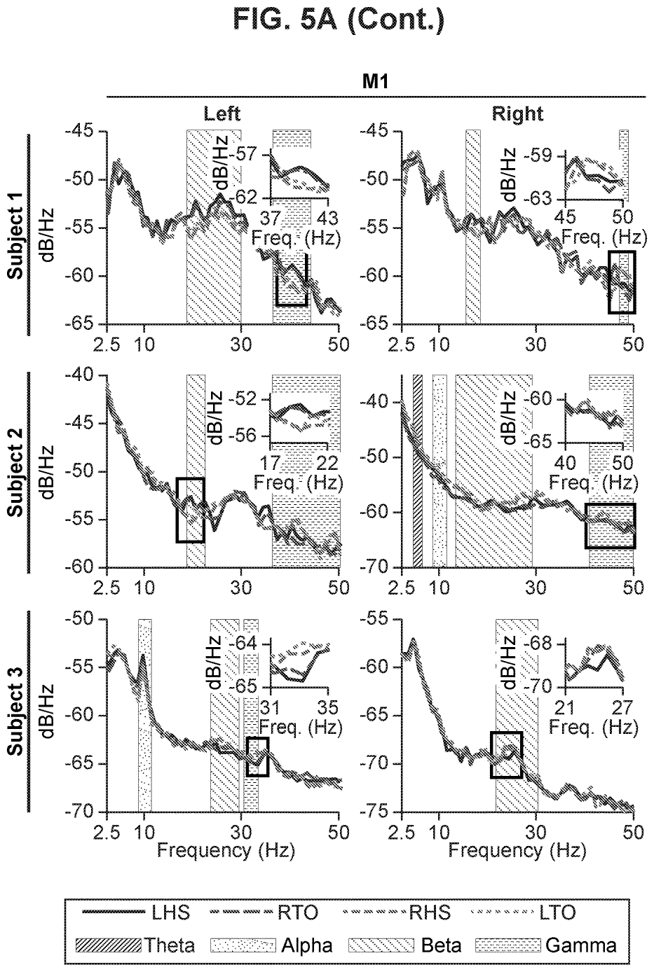

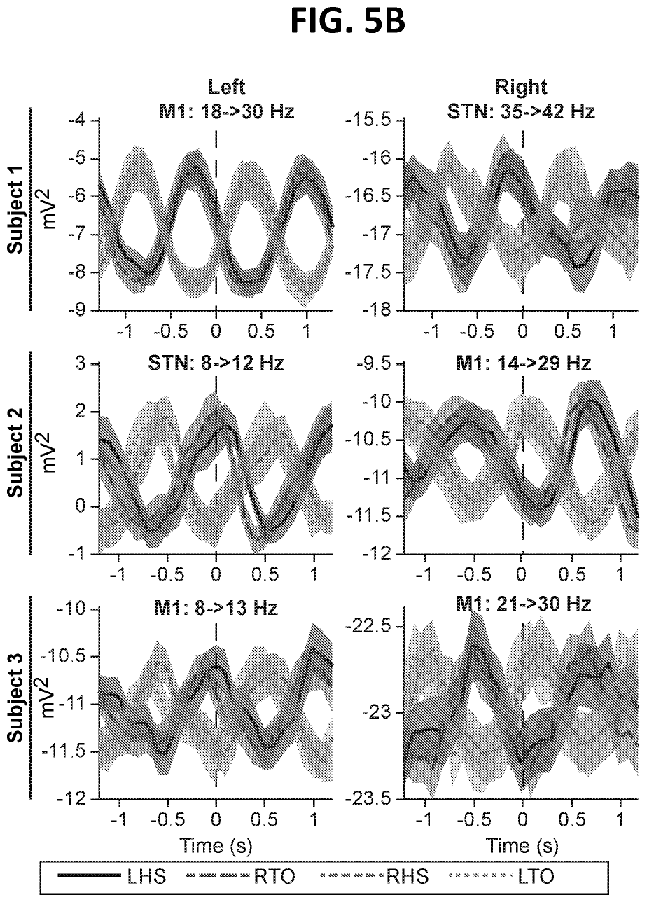

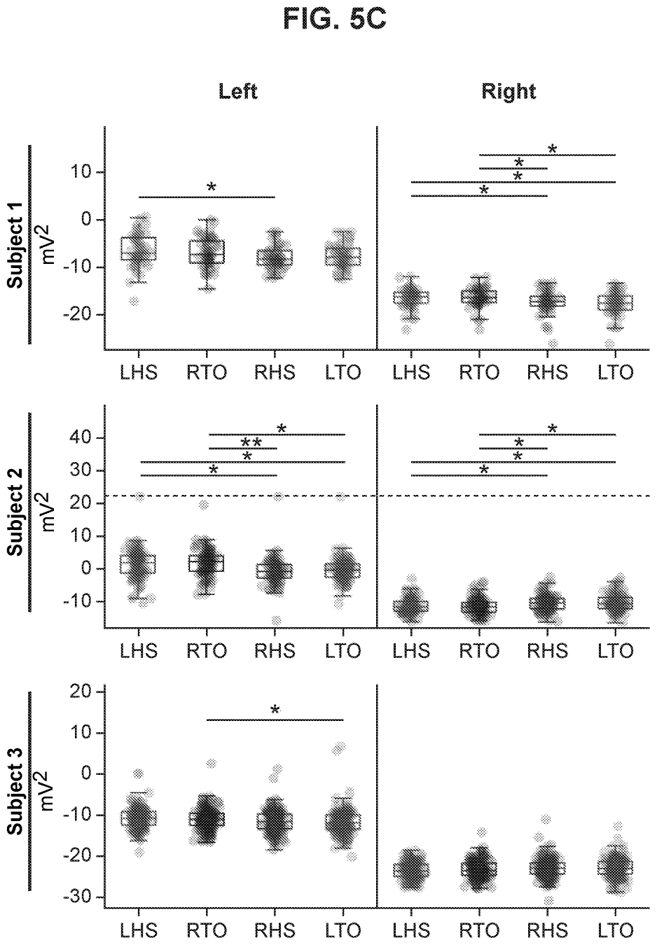

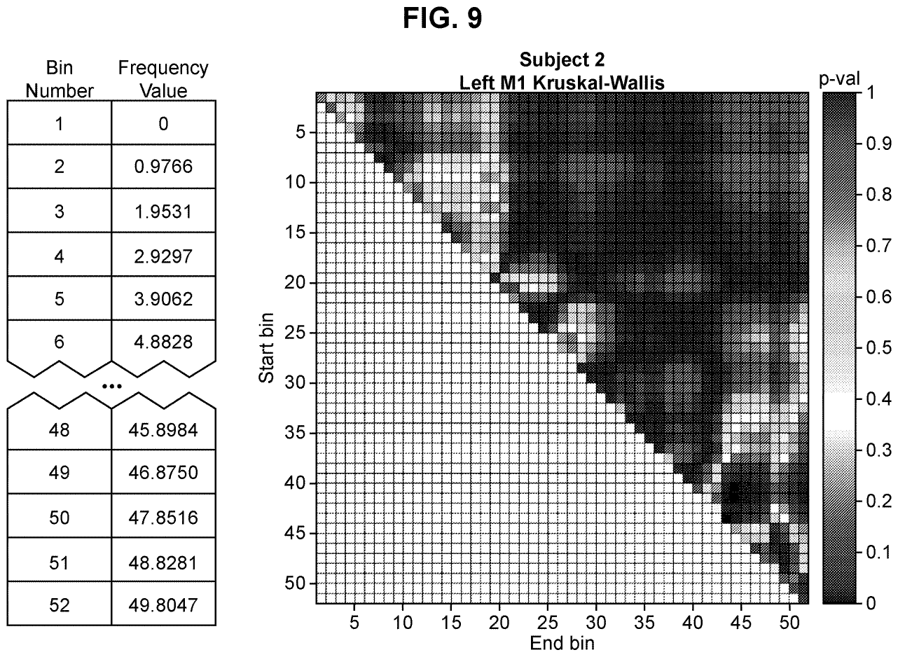

Because the data showed several distinct gait-related frequency bands of modulation during the gait cycle, a data-driven approach was used to determine individual-specific frequency bands that are putative biomarkers for heel-strike and toe-off events. Frequency bands of varying lengths ranging from 0-50 Hz were created, power spectral density values were extracted at each gait event, and an ANOVA test was performed for each band (FIG. 9). It was found that each patient had unique frequency bands where power values differentiated gait events (FIG. 5). Significant gait-event-modulated frequency bands were found within all canonical frequency bands, with a majority in the theta and beta bands (FIG. 5A). Frequency ranges of the gait-event-modulated bands varied by electrode location but were typically a sub-range of the canonical bands. By comparing the instantaneous power spectral density during each of the four gait events, power differences were found between gait events that are temporally distinct (FIG. 5A, inset plots), whereas gait events occurring in temporal proximity have a more similar power spectra profile (FIG. 5A).

To evaluate how the amplitudes of these gait-specific biomarkers change over the gait cycle, their power was averaged over a 1 second period around each gait event and they were found to fluctuate for the duration of the gait cycle (FIG. 5B). Power averages for the left heel-strike and right toe-off events are offset by half a gait cycle to the right heel-strike and left toe-off events. In all subjects, the left and right hemispheres showed reciprocal power modulations across different contacts.

To investigate whether each gait event's instantaneous powers are distinct from each other, a multiple comparison test was performed between all possible pairs of gait events. Significant power differences were found between left and right heel-strikes in subjects 1 and 2 in both hemispheres (FIG. 5C). Other significant differences occurred between toe-off events (FIG. 5C). Gait events temporally close to each other did not differ in power.

### Decoding Gait Events Based on Cortical and Subcortical LFPs

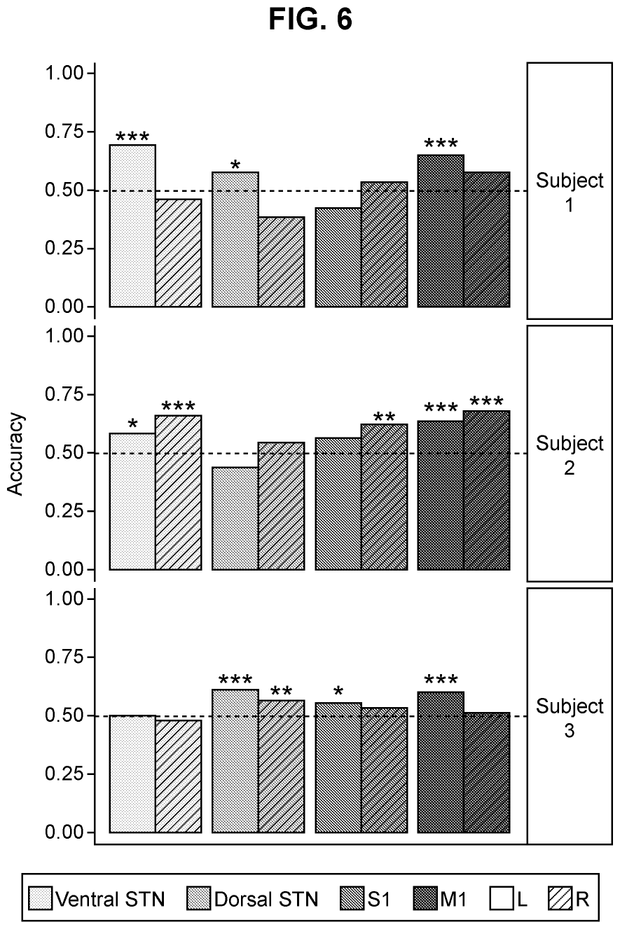

Based on finding spectral signatures for specific gait events of the gait cycle, it was desired to decode gait events using these personalized “gait biomarkers.” Using the linear discriminant analysis (LDA) model, it was possible to classify toe-off events with ≥61% accuracy (Table 2) in all subjects from at least one of the recorded contacts (FIG. 6). Significant above-chance accuracy was achieved from models built using left and right hemisphere data in subjects 2 and 3, but only from left hemisphere trained models in subject 1. No electrode location outperformed others consistently but was subject specific. Overall, the median model accuracies were greater than chance and ranged from 54.4-60.3%. Further analysis of the models showed the maximum discriminatory value achieved, evaluated by calculating the area under the curve (AUC), ranged between 0.585-0.763.

TABLE 2
Classification Summary

| | Median | Maximum | Median | Maximum |
|---|---|---|---|---|
| ID | Accuracy | Accuracy | AUC | AUC |
| Subject 1 | 55.8% | 69.2% | 0.592 | 0.763 |
| Subject 2 | 60.3% | 68.0% | 0.635 | 0.733 |
| Subject 3 | 54.4% | 61.1% | 0.574 | 0.585 |

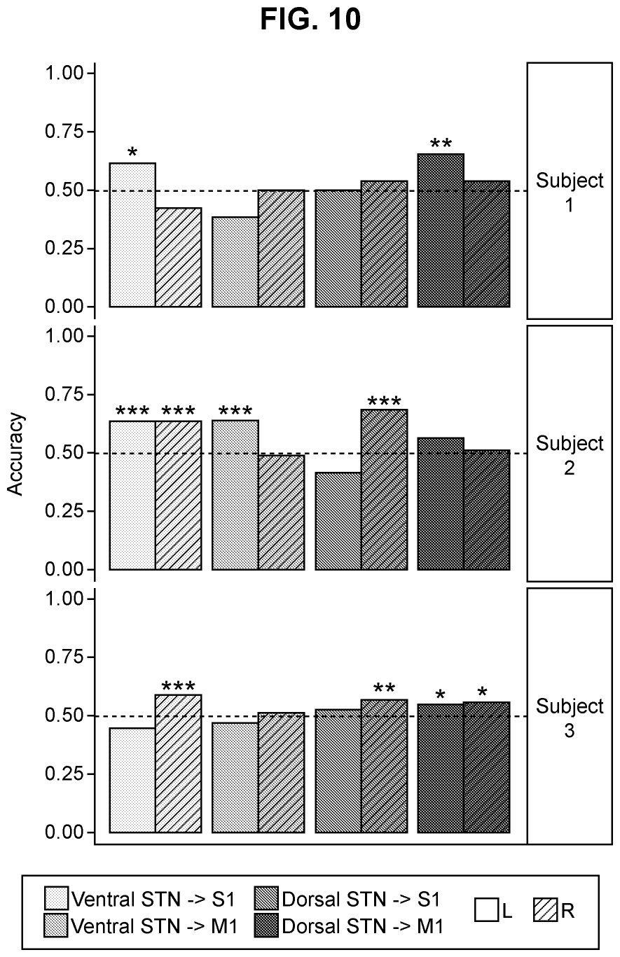

Whether coherence between STN to M1/S1 could classify toe-off events was also explored. The subcortical-cortical coherence pair that achieved the highest accuracy was subject specific, and only subject 2 and 3 had models reach significant above-chance accuracy (accuracy: 58.9-68.3%, AUC: 0.602-0.786) (FIG. 10).

FIGS. 5A to 5C: Unique frequency bands within each subject can differentiate gait events. (A) Average heel-strike and toe-off PSDs from the STN and M1. Each subject had unique frequency bands where power during heel-strikes (left heel-strike=green, right heel-strike=orange) and toe-off (left toe-off=blue, right toe-off=pink) gait events were significantly different (p<0.05). The unique frequency bands were mainly found within the canonical frequency ranges (color of shaded area), but rarely spanned the entire range (width of shaded area). Inset plots show power differences between gait events temporally distinct from each other in relation to the gait cycle. (B) Average power and standard error±1 second around the gait event. Reciprocal power modulation, offset by half a gait cycle, is seen between temporally distinct gait events in all subjects. Furthermore, all left hemisphere data show higher power during left heel-strike/right toe-off and most of the right hemisphere data show higher power during right heel-strike/left toe-off. (C) Boxplot of gait event power within the frequency bands from FIG. 5B. Individual gait event powers are shown as transparent colored dots with outliers shown on the dotted line. Multiple comparison tests were performed against each pair of gait event within the same hemisphere. Level of significance is indicated as follows: *=p<0.05 and **=p<0.005. FIG. 9 shows a visualization of the arbitrary length frequency bands created and an ANOVA p-value heat-map.

FIG. 6: Gait event decoding using oscillatory features achieves greater than chance accuracy. LDA ensemble classifiers were trained on left and right toe-off events for each contact and hemisphere across all subjects. All subjects had at least one contact where at least one model's classification accuracy was ≥61.1%. Maximum accuracy achieved across all subjects was between 61.1-69.2%. Maximum discriminatory ability was calculated using the area under the receiver operator characteristic curve and ranged between 0.585-0.763. Each subject's models are shown on each row. The recorded area the LDA model was built from is indicated in color and follows this order (left to right): red—ventral STN, green—dorsal STN, blue—S1, purple—M1. Bar pattern indicates brain hemisphere the model was built from: solid—left hemisphere, striped—right hemisphere. Asterisks (“*”) above bar indicates significance: p-val<0.05 (*), <0.005 (“**”), <0.0005 (“***”). FIG. 10 shows results from classifier models built using coherence values between the STN and M1.

FIG. 9: Related to FIG. 5. Varying length frequency bands were created between 0-50 Hz. Each frequency is referenced as a bin. Start and end bin refers to the varying length frequency band's start and end frequency. Power during left and right heel-strike and toe-off events were extracted from each frequency band and a Kruskal-Wallis test was performed. The p-value of the Kruskal-Wallis test was stored, and a heat map was created. Example of the resulting heat map is shown from subject 2 M1 recorded area. Significant Kruskal-Wallis test outcomes can be observed to fall within the low gamma band (35-45 Hz) frequency.

FIG. 10: Toe-off gait event decoding using STN-M1 coherence. LDA ensemble classifiers were trained using coherence magnitude squared values between the ventral and dorsal STN to M1 and S1. Highest accuracy and discriminatory value achieved were similar to models built from individual recorded areas. The highest accuracies achieved were between 58.9-68.3% and highest discriminatory values were between 0.602-0.786. Each subject's models are shown on each row. The recorded area the LDA model was built from is indicated in color and follows this order (left to right): pink-ventral STN, yellow-dorsal STN, brown-S1, orange-M1. The bar pattern indicates brain hemisphere the model was built from: solid-left hemisphere, striped-right hemisphere. Asterisks (“*”) above bar indicates significance: p-val<0.05 (*), <0.005 (“**”), <0.0005 (“***”).

### SUMMARY

Chronic invasive recordings were used in PD patients to advance understanding of dynamic subthalamic and sensorimotor oscillatory changes that underlie natural overground walking. First, the novel finding that STN displays increased low frequency (4-12 Hz) activity during the double support period prior to contralateral leg swing was demonstrated. Furthermore, STN shows increased theta frequency coherence with the primary motor during initiation of contralateral leg swing, implicating a potential mechanism for the supraspinal network to scale and fine tune leg muscle activation during stepping. The findings support the hypothesis that oscillations from the basal ganglia and cortex direct alternating power fluctuations between the two hemispheres in that is offset by half a gait cycle, which may indicate a mechanism to coordinate and maintain continuous bipedal locomotion in humans. In addition, patient-specific, gait-related biomarkers were identified in both subcortical and cortical areas at discrete frequency bands. Exploratory ensemble classification models showed above-chance accuracy in classifying left and right gait events using oscillatory power features.

The results of the above experiments provide new insights on the role that subthalamic and sensorimotor oscillations play in human gait. The data also supports the notion that the STN and sensorimotor cortices contain patient-specific, gait-related frequency modulations that can be used to distinguish between left and right gait events. This knowledge has the potential to be integrated into adaptive neuromodulation therapies to improve gait functions in patients with Parkinson's disease.

Alternating Multi-Frequency Modulations from Bilateral STNs During Gait

It was found that the gait-event related alternating power modulations between the left and right STNs during the gait cycle in Parkinson's disease patients are not limited to the high beta frequency range but also involve other low frequency bands.

Based on the results, subthalamic lower frequency (theta and alpha) modulations (i.e., oscillations) during gait were posited to emerge from the STN during periods of gait that require greater cortical engagement. Based on increases in STN theta/alpha power found during the transition from double support (both feet on the ground) to single support (ipsilateral leg on the ground) period, it was postulated that these low frequency modulations engage multiple motor cortical areas to generate the appropriate scale and force required during contralateral leg swing to maintain stable single limb support and bipedal locomotion. Based on increases in STN theta/alpha power that were found during the transition from double support (both fect on the ground) to single support (ipsilateral leg on the ground) period, it was postulated that these low frequency modulations engage multiple motor cortical areas to generate the appropriate scale and force required during contralateral leg swing to maintain stable single limb support and bipedal locomotion. These postulations are supported by previous studies on upper extremity movement tasks have that have shown event-related theta/alpha frequency synchronization within the STN at the onset and throughout the duration of a sustained voluntary muscle contraction task [17]. In some cases, the amplitude of these theta/alpha oscillation correlate with the force generated during hand movement [18]. STN theta activity has also been shown to have a role in the cognitive control of movement, such as during sensorimotor conflict and response inhibition [21].

While previous studies suggest that low-frequency modulations during gait may be secondary to movement-related artifacts [22], the results support the conclusion that these low-frequency oscillations reflect physiological signals for several reasons. First, spectral activities that change during the gait cycle are focal in frequency range and are not broadband in nature (FIG. 3). Second, the spectral power changes in left and right STNs are offset by half a gait cycle, unlike in a previous study where both STNs showed concurrent spectral power increases during the gait cycle regardless of laterality [22]. Finally, the dorsal and ventral STN, as well as M1 and S1 contacts connected to the same RC+S show different time-frequency changes from each other during the gait cycle, and hence less likely to reflect artifacts. The results also suggest the dynamic changes of oscillations across different frequency bands may provide a mechanism to coordinate and recruit different cortical and subcortical areas in response to changes in posture, balance, and forward momentum during walking.

Alternating Multi-Frequency Modulations from Bilateral STNs During Gait

The spatiotemporal specificity of field potentials captured by the permanently implanted cortical electrodes indicate distinct interactions between the STN and different cortical areas during gait. Increased STN-S1 coherence was demonstrated in the low frequency ranges (theta-alpha) during the double-support period between ipsilateral heel-strike and contralateral toe-off. STN-M1 theta frequency coherence was also found to increase during contralateral toe-off and early contralateral leg swing. These alternations in coherence are offset by half a gait cycle between the left and right hemispheres. This is likely the first report of distinct patterns of STN-S1 and STN-M1 synchrony during human gait. It is speculated that increased STN-S1 coherence during ipsilateral heel-strike to contralateral toe-off may represent sensory integration during the double support period as one prepares for leg swing. Increases in STN-M1 theta coherence then follows, during initiation of contralateral leg swing, which may allow the motor cortex to regulate the force of leg muscle activation required to drive forward stepping during gait. While these M1-STN interactions may represent normal recruitment of leg muscles during weight acceptance and transfer phase of the gait cycle, they may also represent compensatory mechanisms by which greater cortical activity is required to drive and maintain locomotion in Parkinson's disease.

### Gait Event Decoding and Potential Clinical Significance

A key finding from the above experiments was that for each patient, a unique range of frequencies were significantly differentially modulated corresponding to the various gait events. While these frequency bands often overlap canonical bands, they are usually narrower and span many different canonical frequencies. The variations among patients may be due to slight differences in electrode placement. The results demonstrate the feasibility of distinguishing gait events based on cortical or STN LFP power. While the results show greater than chance median accuracy and acceptable to medium discriminatory ability, the models may be further optimized for each subject. By unconstraining the set of possible hyperparameter values, possible values that would result in better accuracy and discriminatory ability for different subjects may be discovered. Additionally, techniques and methods may be utilized to correct for overfitting.

One of the reasons to identify gait-specific biomarkers is to use them as control signals for closed-loop, also known as adaptive DBS (aDBS). The Summit RC+S system implanted in the subjects allow for aDBS in real time and utilizes LDA to detect different brain states using Fourier transform power within a frequency band [7]. The aDBS feature of the Summit RC+S device has been successfully tested in PD patients [1] and a cervical dystonia patient [25], with varying timescale for stimulation changes (from 100s of milliseconds to minutes). Therefore, the results demonstrate it is feasible to implement real time aDBS to rapidly change stimulation parameters to improve gait function in Parkinson's disease patients.

### Additional Patient Data

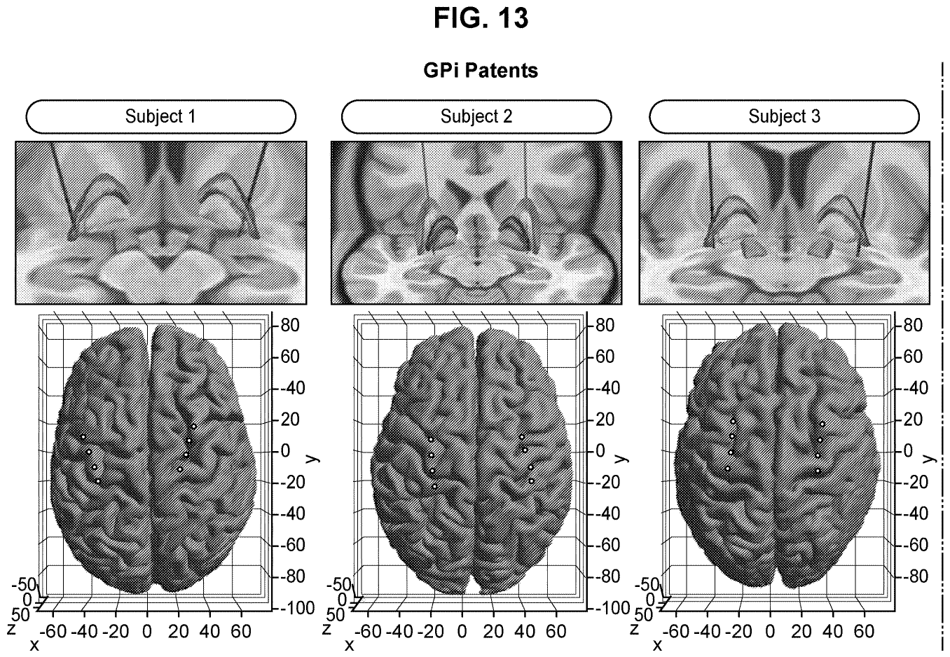

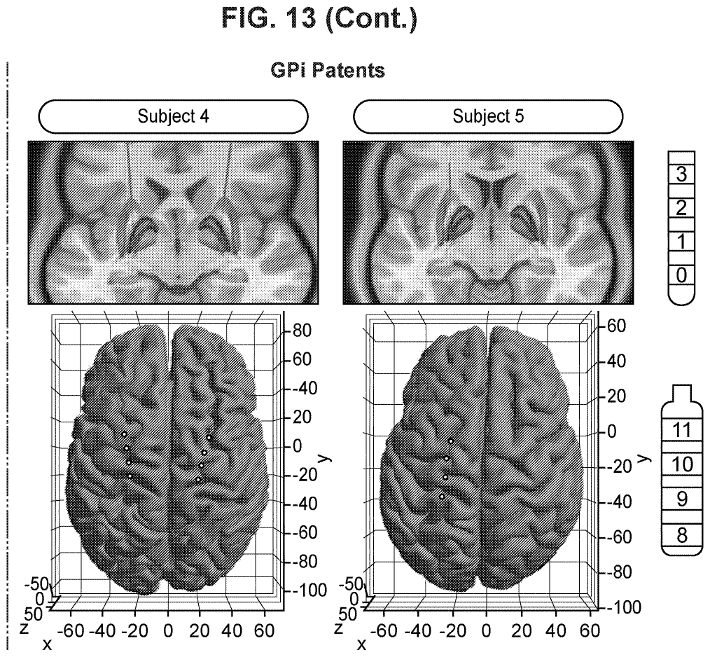

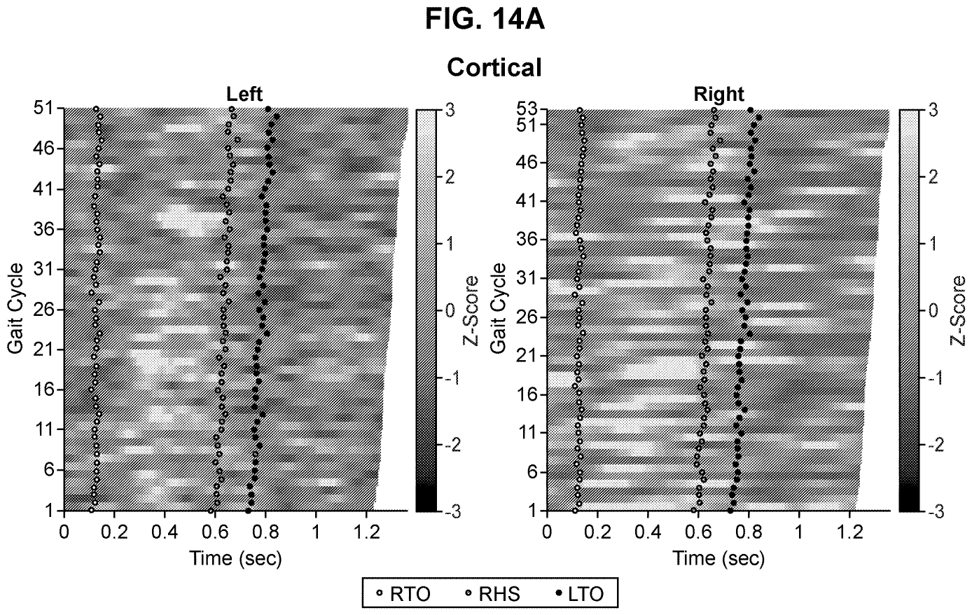

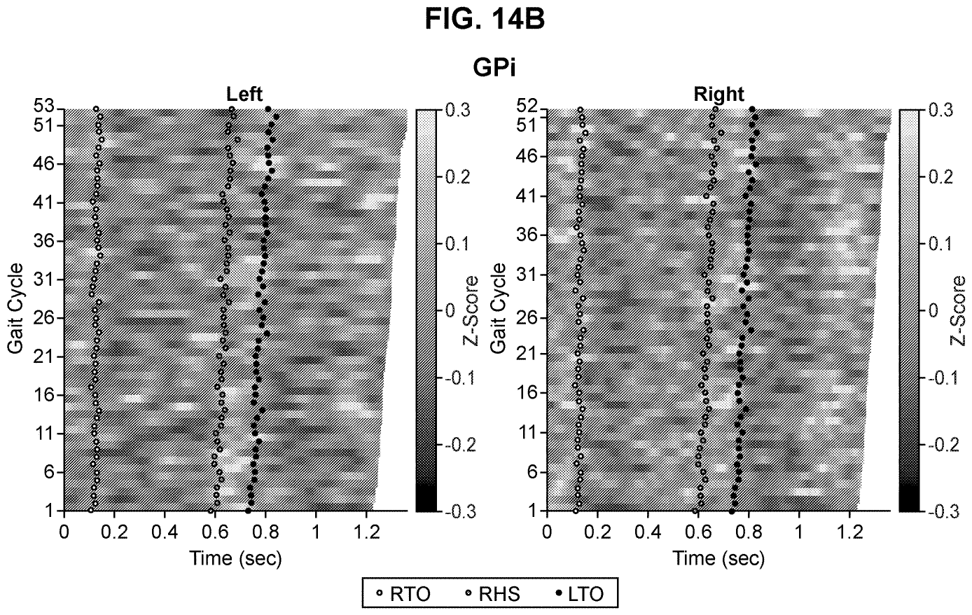

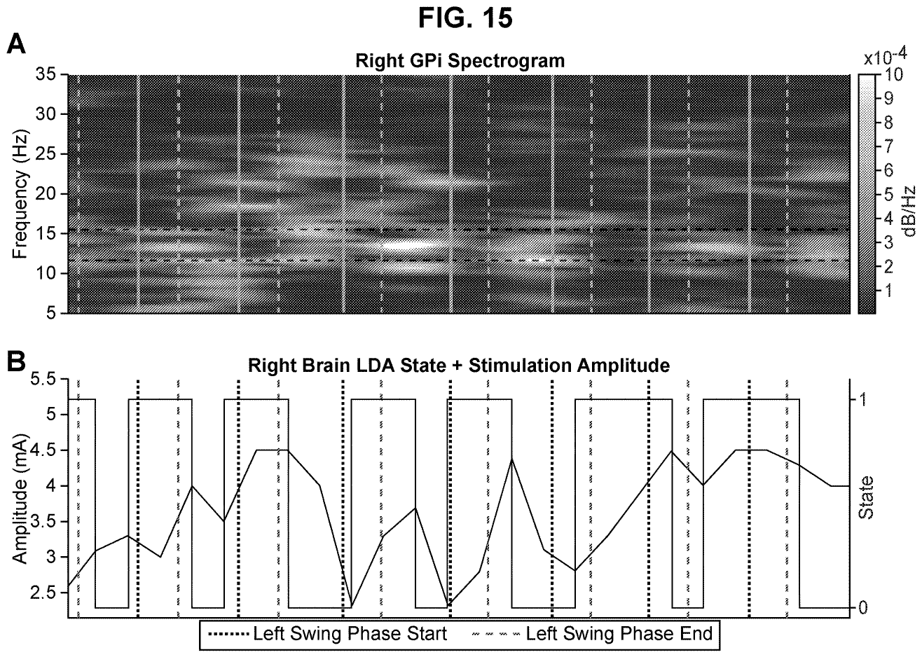

In a similar manner as above, five patients underwent bilateral or unilateral implantation of DBS leads targeting the globus pallidus internus (GPi), and quadripolar cortical paddle electrodes overlying the primary motor (M1) and premotor area (PM) (FIG. 13). Results of experiments performed for the GPi patients can be found in FIGS. 14A to 14B and FIGS. 15A to 15B, and are described below.

FIG. 12: Subcortical and cortical lead reconstructions of five GPi patients. Top row: Deep brain stimulation leads targeting the globus pallidus internus (GPi). Subjects 1-4 are bilaterally implanted, while Subject 5 is unilaterally implanted. Bottom row: Quadripolar cortical paddle electrodes overlying the primary motor (M1) and premotor area (PM).

FIGS. 14A to 14B: Example of gait phase-specific modulation in the GPi and cortical regions from Subject 2. Plots show gait cycles ordered from shortest to longest (top to bottom). Time 0 indicates left heel strike, with other gait events shown as dots: right toe-off (RTO, pink), right heel strike (RHS, orange), and left toe-off (LTO, blue). The spectrogram shows z-scored power values normalized to the entire walking period. Top row: The GPi shows increased modulation in the high beta (20-30 Hz; left) and in the low gamma (30-50 Hz; right) bands during weight-shifting phases. Bottom row: Increased modulation during the right leg swing phase is seen in the high beta (20-30 Hz; primary motor cortex; left) and low beta (13-20 Hz; premotor cortex; right) bands.

FIGS. 15A to 15B: Example of adaptive deep brain stimulation therapy in Subject 2. Adaptive settings were programmed and embedded on the patient's pulse generator. (A) Spectrogram of the patient's neural data originating from the GPi. Dashed lines between 12-15.5 Hz indicate the patient-specific frequency range, determined using an embodiment of the process outlined in the above disclosure, to detect the left leg swing phase. Orange lines indicate the start (solid) and end (dashed) of the left leg swing phase. (B) Pulse generator amplitude and state detection in real-time. The solid black line shows the amplitude increase and decrease depending on the current state of the pulse generator. The gray line shows the detector state of the pulse generator; “state 0” indicates that the left leg is not swinging forward, while “state 1” indicates that the left leg is swinging forward. Orange lines indicate the start (solid) and end (dashed) of the left leg swing phase.

### REFERENCES

[1] Gilron R et al. (2021a) Long-term wireless streaming of neural recordings for circuit discovery and adaptive stimulation in individuals with Parkinson's disease. Nat Biotechnol.
[2] Ewert S, Plettig P, Li N, Chakravarty M M, Collins D L, Herrington T M, Kühn A A, Horn A (2018) Toward defining deep brain stimulation targets in MNI space: A subcortical atlas based on multimodal MRI, histology and structural connectivity. Neuroimage 170:271-282.
[3] Horn A, Kühn A A (2015) Lead-DBS: a toolbox for deep brain stimulation electrode localizations and visualizations. Neuroimage 107:127-135.
[4] Dale A M, Fischl B, Sereno M I (1999) Cortical surface-based analysis. I. Segmentation and surface reconstruction. Neuroimage 9:179-94.
[5] Saad Z S, Reynolds R C, Argall B, Japee S, Cox R W (2004) SUMA: An interface for surface-based intra- and inter-subject analysis with AFNI In: 2004 2nd IEEE International Symposium on Biomedical Imaging: Macro to Nano (IEEE Cat No. 04EX821), pp 1510-1513. IEEE.
[6] Papademetris X, Jackowski M P, Rajeevan N, Okuda H, Constable R T, Staib L H (n.d.) BioImage Suite: An integrated medical image analysis suite, Section of Bioimaging Sciences, Dept. of Diagnostic Radiology, Yale School of Medicine. http://www.bioimagesuite.org.
[7] Sellers K K, Gilron R, Anso J, Louie K H, Shirvalkar P R, Chang E F, Little S J, Starr P A (2021) Analysis-rcs-data: Open-Source Toolbox for the Ingestion, Time-Alignment, and Visualization of Sense and Stimulation Data From the Medtronic Summit RC+S System. Front Hum Neurosci 15.
[8] Polikar R (2006) Ensemble based systems in decision making. IEEE Circuits Syst Mag 6:21-45.
[9] Wolpert D H (1992) Stacked generalization. Neural Networks 5:241-259.
[10] Chen R-C, Dewi C, Huang S-W, Caraka R E (2020) Selecting critical features for data classification based on machine learning methods. J Big Data 7:52.
[11] Genuer R, Poggi J-M, Tuleau-Malot C (2010) Variable selection using random forests. Pattern Recognit Lett 31:2225-2236.
[12] Kuhn M, Wickham H (2020) Tidymodels: a collection of packages for modeling and machine learning using tidyverse principles.
[13] Wright M N, Ziegler A (2017) ranger: A Fast Implementation of Random Forests for High Dimensional Data in C++ and R. J Stat Softw 77.
[14] Altmann A, Tolosi L, Sander O, Lengauer T (2010) Permutation importance: a corrected feature importance measure. Bioinformatics 26:1340-1347.
[15] Herrojo Ruiz M, Rusconi M, Brücke C, Haynes J-D, Schönecker T, Kühn A A (2014) Encoding of sequence boundaries in the subthalamic nucleus of patients with Parkinson's disease. Brain 137:2715-2730.
[16] Kato K, Yokochi F, Iwamuro H, Kawasaki T, Hamada K, Isoo A, Kimura K, Okiyama R, Taniguchi M, Ushiba J (2016) Frequency-Specific Synchronization in the Bilateral Subthalamic Nuclei Depending on Voluntary Muscle Contraction and Relaxation in Patients with Parkinson's Disease. Front Hum Neurosci 10:131.
[17] Tan H, Pogosyan A, Anzak A, Ashkan K, Bogdanovic M, Green A L, Aziz T, Foltynie T, Limousin P, Zrinzo L, Brown P (2013) Complementary roles of different oscillatory activities in the subthalamic nucleus in coding motor effort in Parkinsonism. Exp Neurol 248:187-95.
[18] Anzak A, Tan H, Pogosyan A, Foltynie T, Limousin P, Zrinzo L, Hariz M, Ashkan K, Bogdanovic M, Green A L, Aziz T, Brown P (2012) Subthalamic nucleus activity optimizes maximal effort motor responses in Parkinson's disease. Brain 135:2766-78.
[19] Aron A R, Herz D M, Brown P, Forstmann B U, Zaghloul K (2016) Frontosubthalamic Circuits for Control of Action and Cognition. J Neurosci 36:11489-11495.
[20] Zavala B, Damera S, Dong J W, Lungu C, Brown P, Zaghloul K A (2017) Human Subthalamic Nucleus Theta and Beta Oscillations Entrain Neuronal Firing During Sensorimotor Conflict. Cereb Cortex 27:496-508.
[21] Alegre M, Lopez-Azcarate J, Obeso I, Wilkinson L, Rodriguez-Oroz M C, Valencia M, Garcia-Garcia D, Guridi J, Artieda J, Jahanshahi M, Obeso J A (2013) The subthalamic nucleus is involved in successful inhibition in the stop-signal task: a local field potential study in Parkinson's disease. Exp Neurol 239:1-12.
[22] Hell F, Plate A, Mehrkens J H, Bötzel K (2018) Subthalamic oscillatory activity and connectivity during gait in Parkinson's disease. NeuroImage Clin 19:396-405.
[23] Ansó J, Benjaber M, Parks B, Parker S, Oehrn C R, Petrucci M, Gilron R, Little S, Wilt R, Bronte-Stewart H, Gunduz A, Borton D, Starr P A, Denison T (2022) Concurrent stimulation and sensing in bi-directional brain interfaces: a multi-site translational experience. J Neural Eng 19.
[24] Gilron R, Little S, Wilt R, Perrone R, Anso J, Starr P A (2021b) Sleep-Aware Adaptive Deep Brain Stimulation Control: Chronic Use at Home With Dual Independent Lincar Discriminate Detectors. Front Neurosci 15:732499.
[25] Johnson V, Wilt R, Gilron R, Anso J, Perrone R, Beudel M, Piña-Fuentes D, Saal J, Ostrem J L, Bledsoe I, Starr P, Little S (2021) Embedded adaptive deep brain stimulation for cervical dystonia controlled by motor cortex theta oscillations. Exp Neurol 345:113825.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims. The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

What is claimed is:

1. A method of identifying neural activity biomarkers of an alternating bilateral movement in a subject, the method comprising:
positioning a neural recording device comprising one or more electrodes at a location in a sensorimotor cortex region or basal ganglia system of the brain of the subject to record brain electrical signal data associated with the subject performing the alternating bilateral movement;
instructing the subject to perform an activity comprising the alternating bilateral movement;
recording the brain electrical signal data associated with the movement performed by the subject using the neural recording device, wherein the brain electrical signal data is transmitted to a processor;
recording one or more body parts of the subject performing the movement using a sensor, wherein the movement data recorded by the sensor is transmitted to the processor;
processing the brain electrical signal data and the movement data using the processor, wherein the processing comprises synchronizing the brain electrical signal data and the movement data; and
identifying one or more neural activity biomarkers of the movement in the subject from the processed brain electrical signal data and movement data using the processor, wherein the processor is programmed to use a machine learning algorithm for the identification.

2. The method of claim 1, wherein the subject has a neurological disorder affecting movement.

3. The method of claim 2, wherein the neurological disorder is essential tremor, Parkinson's disease, and/or dystonia.

4. The method of claim 3, wherein the neurological disorder is Parkinson's disease.

5. The method of claim 4, wherein the subject is on medication to treat the Parkinson's disease.

6. The method of claim 5, wherein the medication is a dopaminergic medication.

7. The method according to any of the preceding claims, wherein movement is repeated 5 or more times.

8. The method according to claim 7, wherein the movement is repeated 10 or more times.

9. The method according to claim 7 or 8, wherein the movement comprises the repeated abduction, adduction, flexion, extension, and/or circumduction of one or more body parts of the subject.

10. The method according to claim 9, wherein the movement comprises the repeated flexion and extension of one or more body parts of the subject.

11. The method according to claim 10, wherein the one or more body parts comprises the subjects arms and/or legs.

12. The method according to claim 11, wherein the movement comprises walking.

13. The method according to any of the preceding claims, wherein the sensor is configured to record one or more of: the acceleration or velocity of the one or more body parts; the force generated by the one or more body parts; and the angle between the one or more body parts and a reference body part.

14. The method according to claim 13, wherein the sensor comprises a wearable sensor.

15. The method according to claim 14, wherein the wearable sensor comprises an accelerometer, a force sensitive resistor and/or a goniometer.

16. The method according to claim 13, wherein the sensor comprises an image sensor.

17. The method according to claim 16, wherein the subject wears a motion tracking marker.

18. The method according to any of the preceding claims, wherein the sensor is comprises an electromyography sensor.

19. The method according to claim 18, wherein the electromyography sensor is a surface electromyography sensor.

20. The method according to any of the preceding claims, wherein the neural recording device comprises two or more electrodes.

21. The method according to claim 20, wherein one or more of the electrodes are positioned at a location in a sensorimotor cortex region of the brain and the basal ganglia system of the brain.

22. The method according to claim 20 or 21, wherein one or more of the electrodes are positioned at a location in both the left and right sensorimotor cortex region of the brain and/or both the left and right basal ganglia system of the brain.

23. The method according to any of claims 20 to 22, wherein the sensorimotor cortex region of the brain comprises the precentral gyrus.

24. The method according to claim 23, wherein the precentral gyrus comprises the hand knob area of the precentral gyrus.

25. The method according to any of claims 20 to 24, wherein the sensorimotor cortex region of the brain comprises the somatosensory cortex.

26. The method according to any of claims 20 to 25, wherein the basal ganglia system comprises the subthalamic nucleus.

27. The method according to any of the preceding claims, wherein the neural recording device comprises a deep brain stimulation lead configured to be positioned in the basal ganglia system of the brain.

28. The method according to claim 23, wherein the neural recording device comprises bilateral leads.

29. The method according to any of the preceding claims, wherein the neural recording device comprises an electrocorticography (ECOG) paddle configured to be positioned in the sensorimotor cortex region of the brain.

30. The method according to claim 29, wherein the neural recording device comprises bilateral paddles.

31. The method according to any of claims 27 to 30, wherein the brain electrical signal data is wirelessly transmitted to the processor using an implantable pulse generator.

32. The method according to any of the preceding claims, wherein the processing further comprises preprocessing the brain electrical signal data using a high-pass and/or low-pass filter.

33. The method according to any of the preceding claims, wherein electrical signal data comprises neural oscillations in a range from 4 Hz to 30 Hz.

34. The method according to any of the preceding claims, wherein the processing further comprises calculating one or more of a continuous wavelet transform (CWT), wavelet coherence, short-time Fourier transform (STFT), and power spectral density (PSD) using the synchronized brain electrical signal data and the movement data.

35. The method according to any of the preceding claims, wherein the machine learning algorithm comprises a Random Forest (RF) algorithm.

36. The method according to claim 35, wherein the processed brain electrical signal data and movement data and the RF algorithm are used to train a machine learning model for identifying brain electrical signal data features of neural activity biomarkers.

37. The method according to claim 36, wherein the RF trained machine learning model is one of multiple ensemble models used for alternating bilateral movement event classification.

38. The method according to claim 37, wherein the machine learning algorithm comprises one or more of a K-nearest neighbors (KNN), logistic regression, linear discriminant analysis (LDA), Gradient Boosted Decision Trees (XGBoost), and neural network algorithm for training a machine learning model of the ensemble of models for event classification.

39. The method according to claim 37 or 38, wherein the trained ensemble of machine learning models is used to generate control signals for real time adaptive deep brain stimulation.

40. The method according to any of claims 35 to 39, wherein the identified neural activity biomarkers of alternating bilateral movement are used to generate control signals for real time adaptive deep brain stimulation.

41. A system for identifying neural activity biomarkers of alternating bilateral movement in a subject configured to perform the method according to any of claims 1 to 40.

42. A system for identifying neural activity biomarkers of alternating bilateral movement in a subject, the system comprising:
a neural recording device comprising an electrode adapted for positioning at a location in a sensorimotor cortex region or basal ganglia system of the brain of the subject to record brain electrical signal data associated with the subject performing the alternating bilateral movement;
a sensor configured to record one or more body parts of the subject performing the movement;
a processor configured to receive the brain electrical signal data from the neural recording device and the movement data from the sensor; and
memory operably coupled to the processor wherein the memory comprises instructions stored thereon, which when executed by the processor, cause the processor to process the brain electrical signal data and the movement data by synchronizing the brain electrical signal data and the movement data, and identify one or more neural activity biomarkers of the movement in the subject from the processed brain electrical signal data and movement data using a machine learning algorithm.

43. The system according to claim 42, wherein the sensor is configured to record one or more of: the acceleration or velocity of the one or more body parts; the force generated by the one or more body parts; and the angle between the one or more body parts and a reference body part.

44. The system according to claim 43, wherein the sensor comprises a wearable sensor.

45. The system according to claim 44, wherein the wearable sensor comprises an accelerometer, a force sensitive resistor and/or a goniometer.

46. The system according to claim 42 or 43, wherein the sensor comprises an image sensor.

47. The system according to claim 46, wherein the subject wears a motion tracking marker.

48. The system according to any of the claims 42 to 44, wherein the sensor is comprises an electromyography sensor.

49. The system according to claim 48, wherein the electromyography sensor is a surface electromyography sensor.

50. The system according to any of claims 42 to 49, wherein the neural recording device comprises two or more electrodes.

51. The system according to claim 50, wherein one or more of the electrodes are adapted for positioning at a location in a sensorimotor cortex region of the brain and the basal ganglia system of the brain.

52. The system according to claim 50 or 51, wherein one or more of the electrodes are adapted for positioning at a location in both the left and right sensorimotor cortex region of the brain and/or both the left and right basal ganglia system of the brain.

53. The system according to any of claims 50 to 52, wherein the sensorimotor cortex region of the brain comprises the precentral gyrus.

54. The system according to claim 53, wherein the precentral gyrus comprises the hand knob area of the precentral gyrus.

55. The system according to any of claims 50 to 54, wherein the sensorimotor cortex region of the brain comprises the somatosensory cortex.

56. The system according to any of claims 50 to 55, wherein the basal ganglia system comprises the subthalamic nucleus.

57. The system according to any of claims 42 to 56, wherein the neural recording device comprises a deep brain stimulation lead adapted for positioning in the basal ganglia system of the brain.

58. The system according to claim 57, wherein the neural recording device comprises bilateral leads.

59. The system according to any of claims 42 to 58, wherein the neural recording device comprises an electrocorticography (ECoG) paddle adapted for positioning in the sensorimotor cortex region of the brain.

60. The system according to claim 59, wherein the neural recording device comprises bilateral paddles.

61. The system according to any of claims 42 to 60, wherein the system further comprises an implantable pulse generator configured to wirelessly transmit the brain electrical signal data to the processor.

62. The system according to any of claims 42 to 61, wherein the processing further comprises preprocessing the brain electrical signal data using a high-pass and/or low-pass filter.

63. The system according to any of claims 42 to 62, wherein the electrical signal data comprises neural oscillations in a range from 4 Hz to 30 Hz.

64. The system according to any of claims 42 to 63, wherein the processing further comprises calculating one or more of a continuous wavelet transform (CWT), wavelet coherence, short-time Fourier transform (STFT), and power spectral density (PSD) using the synchronized brain electrical signal data and the movement data.

65. The system according to any of claims 42 to 64, wherein the machine learning algorithm comprises a Random Forest (RF) algorithm.

66. The system according to claim 65, wherein the memory comprises instructions stored thereon, which when executed by the processor, cause the processor to train a machine learning model for identifying brain electrical signal data features of neural activity biomarkers using the processed brain electrical signal data and movement data and the RF algorithm.

67. The system according to claim 66, wherein the RF trained machine learning model is one of multiple ensemble models used for alternating bilateral movement event classification.

68. The system according to claim 67, wherein the memory comprises instructions stored thereon, which when executed by the processor, cause the processor to train a machine learning model of the ensemble of models for event classification using the processed brain electrical signal data and movement data and one or more of a K-nearest neighbors (KNN), logistic regression, linear discriminant analysis (LDA), Gradient Boosted Decision Trees (XGBoost), and neural network algorithm.

69. The system according to claim 67 or 68, wherein the memory comprises instructions stored thereon, which when executed by the processor, cause the processor to generate control signals for real time adaptive deep brain stimulation using recorded brain electrical signal data and the trained ensemble of machine learning models.

70. The system according to any of claims 65 to 69, wherein the memory comprises instructions stored thereon, which when executed by the processor, cause the processor to generate control signals for real time adaptive deep brain stimulation using the identified neural activity biomarkers of alternating bilateral movement in the subject.

71. A kit comprising the system of any of claims 42 to 70 and instructions for identifying neural activity biomarkers of alternating bilateral movement in a subject.
